(19) United States
(12) Patent Application Publication
(10) Pub. No.: US 2005/0054022 A1
(43) Pub. Date: Mar. 10, 2005

(54) Methods and compositions comprising FATP5 for use in the diagnosis and treatment of metabolic disorders

(75) Inventors: Ruth E. Gimeno, Wellesley, MA (US); Brian K. Hubbard, Beverly, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/919,608

(22) Filed: Aug. 17, 2004

Related U.S. Application Data
(60) Provisional application No. 60/496,098, filed on Aug. 18, 2003.

(51) Int. Cl. G01N 33/53; G01N 33/537; G01N 33/543

(52) U.S. Cl. 435/7.92

(57) ABSTRACT

The present invention provides methods for the identification of agents, e.g., therapeutic agents, that inhibit Fatty Acid Transport 5 (FATP5) activity, and methods of treating diseases or conditions associated with FATP5 function, e.g., obesity, insulin resistance, type 2 diabetes, dyslipidemia, fatty liver disease, and cardiovascular disease. Further aspects of the invention provide a transgenic FATP5 non-human knockout mammal, e.g., mouse, useful for elucidating the function of FATP5 in intact animals whose genomes comprise a wild-type FATP5 gene.

### CROSS-REFERENCES TO RELATED APPLICATION

This application claims the benefit of U.S. Provisional Application No. 60/496,098, filed Aug. 18, 2003, the contents of which are incorporated herein by this reference.

### BACKGROUND OF THE INVENTION

Obesity represents the most prevalent of body weight disorders, affecting an estimated 30 to 50% of the middle-aged population in the western world. Obesity, defined as a body mass index (BMI) of 30 kg/m² or more, contributes to diseases such as coronary artery disease, hypertension, stroke, diabetes, hyperlipidemia and some cancers. (See, e.g., Nishina, P. M. et al. (1994), Metab. 43:554-558; Grundy, S. M. & Barnett, J. P. (1990), Dis. Mon. 36:641-731). Obesity is a complex multifactorial chronic disease that develops from an interaction of genotype and the environment and involves social, behavioral, cultural, physiological, metabolic and genetic factors.

Non-insulin dependent diabetes mellitus(NIDDM) or Type 2 diabetes is the most common metabolic disease worldwide. Every day, 1700 new cases of diabetes are diagnosed in the United States, and at least one-third of the 16 million Americans with diabetes are unaware of it. Diabetes is the leading cause of blindness, renal failure, and lower limb amputations in adults and is a major risk factor for cardiovascular disease and stroke. Type 2 diabetes is the form of diabetes characterized by insulin resistance combined with a relative, rather than an absolute, deficiency of insulin. Insulin resistance can be defined as a diminished ability of insulin to exhibit effective biological activity over a range of concentrations. Individuals suffering from Type 2 diabetes manifest considerable variation in the degree of each of the two defects, from demonstrating predominantly insulin resistance with minimal insulin deficiency to demonstrating predominantly insulin deficiency with minimal insulin resistance.

Normal glucose homeostasis requires the finely tuned orchestration of insulin secretion by pancreatic beta cells in response to subtle changes in blood glucose levels, delicately balanced with secretion of counter-regulatory hormones such as glucagon. One of the fundamental actions of insulin is to stimulate uptake of glucose from the blood into tissues, especially muscle and fat. Type 2 or non-insulin-dependent diabetes mellitus (NIDDM) accounts for >90% of cases and is characterized by a triad of (1) resistance to insulin action on glucose uptake in peripheral tissues, especially skeletal muscle and adipocytes, (2) impaired insulin action to inhibit hepatic glucose production, and (3) misregulated insulin secretion (DeFronzo, (1997) Diabetes Rev. 5:177-269). In most cases, type 2 diabetes is a polygenic disease with complex inheritance patterns (reviewed in Kahn et al., (1996) Annu. Rev. Med. 47:509-531).

Environmental factors, especially diet, physical activity, and age, interact with genetic predisposition to affect disease prevalence. Studies indicate that the combination of insulin resistance, obesity and elevated plasma levels of free fatty acids are common in both individuals “at risk” for developing Type 2 diabetes and those with clinical symptoms of the disease (Boden, G., 1999, Proc. Assoc. Am. Physicians, 111:241-248). Susceptibility to both insulin resistance and insulin secretory defects appears to be genetically determined (Kahn, et al.). Defects in insulin action precede the overt disease and are seen in non-diabetic relatives of diabetic subjects. Therefore, it would be extremely useful to understand the role these factors play in the development and pathology of Type 2 diabetes. In spite of intense investigation, the genes responsible for the common forms of Type 2 diabetes remain unknown.

### DESCRIPTION OF THE INVENTION

The present invention is based, at least in part, on the discovery that transgenic animals deficient for FATP5 are protected from diet induced obesity and insulin resistance. Still further, FATP5 deficient animals display a beneficial plasma lipid profile. Thus, the present invention provides methods for identifying compounds capable of modulating FATP5 associated disorders, e.g., metabolic disorders, (e.g., obesity, insulin resistance, type II diabetes, dislipidemia), cardiovascular disorders (e.g., free fatty acid levels, triglyceride levels, coronary artery disease, hypertension, and stroke), and fatty liver disease. Methods provided include assaying the ability of a compound to modulate FATP5 nucleic acid expression or FATP5 polypeptide activity, including enzyme activity and fatty acid or bile acid uptake activity. Additionally provided are methods for modulating an FATP5 polypeptide activity to thereby affect FATP5 mediated cellular processes and disorders.

In one embodiment the invention provides a method of identifying whether a compound is a candidate compound capable of modulating an FATP5-mediated metabolic disorder, the method comprising, combining a test compound with a composition comprising a FATP5 polypeptide; measuring an acyl-CoA ligase or bile acid CoA ligase activity of the FATP5 polypeptide in the composition in the presence and absence of the test compound; and identifying the test compound as the candidate compound for use in modulating the FATP5-mediated metabolic disorder when the FATP5 polypeptide acyl-CoA ligase or bile acid CoA ligase activity in the presence of the compound differs from the FATP5 polypeptide Acyl-CoA ligase or bile acid CoA ligase activity in the absence of the compound. As used herein, an FATP5-mediated metabolic disorder can be any one of a body weight disorder (e.g., obesity), an insulin resistance disorder (e.g., type 2 diabetes), dyslipidemia (e.g., elevated serum triglyceride levels, elevated free fatty acid levels), cardiovascular disorders (e.g., coronary heart disease, atherosclerosis, hypertension, ischemic heart disease), and fatty liver disease (steatosis). In certain embodiments, the composition comprising the FATP5 polypeptide can be a membrane preparation, a cell expressing an FATP5 polypeptide, or an isolated FATP5 polypeptide.

Another embodiment includes the above method and further includes administering a test compound which has been identified in the described method as modulating FATP5 acyl CoA ligase or bile acid CoA ligase activity to a mammal and determining whether the test compound modulated an FATP5 mediated process; then identifying the test compound which modulates an FATP5 mediated process as a candidate compound useful for modulating an FATP5 mediated disorder. In certain embodiments, the FATP5 mediated process monitored is selected from one or more of body weight, body fat composition, dyslipidemia, feeding behavior, insulin resistance, glucose uptake, fatty acid or bile acid uptake, bile acid composition in bile, feces, urine or plasma, and/or plasma lipid composition.

In certain embodiments throughout the methods provided herein, the FATP5 activity is defined as an acyl-CoA ligase activity. In a further embodiment FATP5 activity is a bile acyl-CoA ligase activity. The methods comprise embodiments wherein acyl CoA ligase activity can be determined by measuring production of phosphate, acyl-CoA, or AMP. Additional embodiments comprise aspects wherein acyl CoA ligase activity can be determined by measuring consumption of coenzyme A, ATP, or fatty acid/bile acid.

In another embodiment, the invention provides a method for identifying a candidate compound useful for modulating an FATP5 mediated disorder comprising, combining a test compound with a composition comprising a FATP5 polypeptide, and determining whether the test compound binds to the FATP5 polypeptide; followed by administering the compound identified as binding to the FATP5 polypeptide to a mammal, and determining whether the test compound modulates an FATP5 mediated process. Thus, identifying a test compound is identified as a candidate compound useful for modulating an FATP5 mediated disorder when the compound modulated an FATP5 mediated process. As used herein, an FATP5-mediated metabolic disorder can be any one of a body weight disorder (e.g., obesity), an insulin resistance disorder (e.g., type 2 diabetes), dyslipidemia (e.g., elevated serum triglyceride levels, elevated free fatty acid levels), cardiovascular disorders (e.g., coronary heart disease, atherosclerosis, hypertension, ischemic heart disease), and fatty liver disease (steatosis). In certain embodiments, the composition comprising the FATP5 polypeptide can be a membrane preparation, a cell expressing an FATP5 polypeptide, a tissue expressing an FATP5 polypeptide, or an isolated FATP5 polypeptide. In additional embodiments, the FATP5 mediated process can be selected from any one or more of: body weight, body fat composition, feeding behavior, insulin resistance, glucose uptake, fatty acid or bile acid uptake, dyslipidemia, plasma lipid composition, and/or bile acid composition in bile, feces, urine, or plasma.

In another embodiment the invention provides a method for identifying a candidate compound useful for modulating an FATP5 mediate disorder comprising combining a test compound with a composition comprising a cell capable of expressing an FATP5 polypeptide; measuring expression of the FATP5 polypeptide in the composition in the presence and absence of the test compound; then identifying the test compound as a candidate compound for use in modulating an FATP5 mediated metabolic disorder when the FATP5 polypeptide expression in the presence of the compound differs from the FATP5 polypeptide in the absence of the compound. The FATP5 mediated metabolic disorder can be any of body weight disorder (e.g., obesity), an insulin resistance disorder (e.g., type 2 diabetes), dyslipidemia (e.g., elevated serum triglyceride levels, elevated free fatty acid levels), cardiovascular disorders (e.g., coronary heart disease, atherosclerosis, hypertension, ischemic heart disease), and fatty liver disease (steatosis). In certain embodiments, FATP5 expression can be measured by monitoring one or more of the transcript levels, protein levels, fatty acid/bile acid uptake, or acyl CoA ligase or bile acid CoA ligase activity.

Still another embodiment provides a method for identifying a candidate compound capable of modulating an FATP5 mediated metabolic disorder comprising combining a test compound and a fatty acid with a cell expressing a FATP5 polypeptide, and measuring uptake of fatty acid or bile acid in the test cell; then identifying a test compound as a candidate compound for use in modulating an FATP5 mediated metabolic disorder when uptake of fatty acid or bile acid in the cell in the presence of the compound differs from the uptake of the fatty acid in the absence of compound. The FATP5 mediated metabolic disorder can be any of body weight disorder (e.g., obesity), an insulin resistance disorder (e.g., type 2 diabetes), dyslipidemia (e.g., elevated serum triglyceride levels, elevated free fatty acid levels), cardiovascular disorders (e.g., coronary heart disease, atherosclerosis, hypertension, ischemic heart disease), and fatty liver disease (steatosis).

In one embodiment the invention provides a method of identifying whether a compound is a candidate compound capable of modulating an FATP5-mediated body weight disorder, the method comprising, combining a test compound with a composition comprising a FATP5 polypeptide; measuring an activity (e.g., acyl-CoA ligase, bile acid ligase activity) of the FATP5 polypeptide in the composition in the presence and absence of the test compound; and identifying the test compound as the candidate compound for use in modulating the FATP5-mediated body weight disorder when the FATP5 polypeptide activity in the presence of the compound differs from the FATP5 polypeptide activity in the absence of the compound. In certain embodiments, the composition comprising the FATP5 polypeptide can be a membrane preparation, a cell expressing an FATP5 polypeptide, or an isolated FATP5 polypeptide.

Another embodiment includes the above method and further includes administering a test compound which has been identified in the described method as modulating FATP5 activity (e.g., acyl CoA ligase activity, bile acid CoA ligase activity) to a mammal and determining whether the test compound modulated an FATP5 mediated body weight process; then identifying the test compound which modulates an FATP5 mediated process as a candidate compound useful for modulating an FATP5 mediated body weight disorder. In certain embodiments, the FATP5 mediated body weight process monitored is selected from one or more of body weight, body fat composition, feeding behavior, insulin resistance, glucose uptake, fatty acid or bile acid uptake, dyslipidemia, plasma lipid composition, and/or bile acid composition in bile, feces, urine or plasma.

In another embodiment, the invention provides a method for identifying a candidate compound useful for modulating an FATP5 mediated body weight disorder comprising, combining a test compound with a composition comprising a FATP5 polypeptide, and determining whether the test compound binds to the FATP5 polypeptide; followed by administering the compound identified as binding to the FATP5 polypeptide to a mammal, and determining whether the test compound modulates an FATP5 mediated body weight process. Thus, a test compound is identified as a candidate compound useful for modulating an FATP5 mediated disorder when the compound modulated an FATP5 mediated body weight process. In certain embodiments, the composition comprising the FATP5 polypeptide can be a membrane preparation, a cell expressing an FATP5 polypeptide, a tissue expressing an FATP5 polypeptide, or an isolated FATP5 polypeptide. In additional embodiments, the FATP5 mediated body weight process can be selected from any one or more of: body weight, body fat composition, feeding behavior, and/or fatty acid or bile acid uptake.

In another embodiment the invention provides a method for identifying a candidate compound useful for modulating an FATP5 mediated body weight disorder comprising combining a test compound with a composition comprising a cell capable of expressing an FATP5 polypeptide; measuring expression of the FATP5 polypeptide in the composition in the presence and absence of the test compound; then identifying the test compound as a candidate compound for use in modulating an FATP5 mediated body weight disorder when the FATP5 polypeptide expression in the presence of the compound differs from the FATP5 polypeptide in the absence of the compound. The FATP5 mediated body weight disorder includes, for example., obesity. In certain embodiments, FATP5 expression can be measured by monitoring one or more of the transcript levels, protein levels, fatty acid/bile acid uptake, or acyl CoA/bile acid CoA ligase activity.

Still another embodiment provides a method for identifying a candidate compound capable of modulating an FATP5 mediated body weight disorder comprising combining a test compound and a fatty/bile acid with a cell expressing a FATP5 polypeptide, and measuring uptake of fatty/bile acid in the test cell; then identifying a test compound as a candidate compound for use in modulating an FATP5 mediated body weight disorder when uptake of fatty/bile acid in the cell in the presence of the compound differs from the uptake of the fatty/bile acid in the absence of compound. The FATP5 mediated body weight disorder can be, for example, obesity.

In one embodiment the invention provides a method of identifying whether a compound is a candidate compound capable of modulating an FATP5-mediated insulin resistance disorder, the method comprising, combining a test compound with a composition comprising a FATP5 polypeptide; measuring an acyl-CoA/bile acid CoA ligase activity of the FATP5 polypeptide in the composition in the presence and absence of the test compound; and identifying the test compound as the candidate compound for use in modulating the FATP5-mediated insulin resistance disorder when the FATP5 polypeptide acyl-CoA/bile acid CoA ligase activity in the presence of the compound differs from the FATP5 polypeptide acyl-CoA/bile acid CoA ligase activity in the absence of the compound. As used herein, an FATP5-mediated insulin resistance disorder includes type 2 diabetes, for example. In certain embodiments, the composition comprising the FATP5 polypeptide can be a membrane preparation, a cell expressing an FATP5 polypeptide, or an isolated FATP5 polypeptide.

Another embodiment includes the above method and further includes administering a test compound which has been identified in the described method as modulating FATP5 acyl CoA/bile acid CoA ligase activity to a mammal and determining whether the test compound modulates insulin resistance; then identifying the test compound which modulates insulin resistance as a candidate compound useful for modulating an FATP5 mediated disorder. In certain embodiments, the insulin resistance can be monitored by detecting whole body or tissue glucose uptake, hepatic glucose output, blood glucose level, or blood insulin level.

In another embodiment, the invention provides a method for identifying a candidate compound useful for modulating an insulin resistance disorder comprising, combining a test compound with a composition comprising a FATP5 polypeptide, and determining whether the test compound binds to the FATP5 polypeptide; followed by administering the compound identified as binding to the FATP5 polypeptide to a mammal, and determining whether the test compound modulates insulin resistance. Thus, identifying a test compound is identified as a candidate compound useful for modulating an insulin resistance disorder when the compound modulates insulin resistance. As used herein, an insulin resistance disorder can include insulin resistance and type 2 diabetes. In certain embodiments, the composition comprising the FATP5 polypeptide can be a membrane preparation, a cell expressing an FATP5 polypeptide, a tissue expressing an FATP5 polypeptide, or an isolated FATP5 polypeptide. In additional embodiments, insulin resistance can be measured by detecting any one or more of: whole body or tissue glucose uptake, hepatic glucose output, blood glucose level, or blood insulin level.

In another embodiment the invention provides a method for identifying a candidate compound useful for modulating an insulin resistance disorder comprising combining a test compound with a composition comprising a cell capable of expressing an FATP5 polypeptide; measuring expression of the FATP5 polypeptide in the composition in the presence and absence of the test compound; then identifying the test compound as a candidate compound for use in modulating an insulin resistance disorder when the FATP5 polypeptide expression in the presence of the compound differs from the FATP5 polypeptide in the absence of the compound. The insulin resistance disorder includes, e.g., type 2 diabetes. In certain embodiments, FATP5 expression can be measured by monitoring one or more of the transcript levels, protein levels, fatty acid or bile acid uptake, or acyl CoA/bile acid CoA ligase activity.

Still another embodiment provides a method for identifying a candidate compound capable of modulating an insulin resistance disorder comprising combining a test compound and a fatty/bile acid with a cell expressing a FATP5 polypeptide, and measuring uptake of fatty/bile acid in the test cell; then identifying a test compound as a candidate compound for use in modulating an insulin resistance disorder when uptake of fatty/bile acid in the cell in the presence of the compound differs from the uptake of the fatty/bile acid in the absence of compound. The insulin resistance disorder includes, e.g., type 2 diabetes.

In one embodiment the invention provides a method of identifying whether a compound is a candidate compound capable of modulating dyslipidemia, the method comprising combining a test compound with a composition comprising a FATP5 polypeptide; measuring an acyl-CoA ligase or bile acid CoA ligase activity of the FATP5 polypeptide in the composition in the presence and absence of the test compound; and identifying the test compound as the candidate compound for use in modulating dyslipidemia when the FATP5 polypeptide ligase activity (e.g., acyl CoA ligase, bile acid CoA ligase activity) in the presence of the compound differs from the FATP5 polypeptide ligase activity (e.g., acyl CoA ligase, bile acid CoA ligase activity) in the absence of the compound. As used herein, dyslipidemia can include, e.g., elevated serum triglyceride levels, elevated free fatty acid levels. In certain embodiments, the composition comprising the FATP5 polypeptide can be a membrane preparation, a cell expressing an FATP5 polypeptide, or an isolated FATP5 polypeptide.

Another embodiment includes the above method and further includes administering a test compound which has been identified in the described method as modulating FATP5 acyl CoA ligase/bile acid CoA ligase activity to a mammal and determining whether the test compound modulates dyslipidemia; then identifying the test compound which modulates dyslipidemia as a candidate compound useful for modulating dyslipidemia. In certain embodiments, dyslipidemia can be monitored by determining free fatty acid levels, serum triglyceride levels, and/or plasma lipid composition.

In another embodiment, the invention provides a method for identifying a candidate compound useful for modulating dyslipidemia comprising, combining a test compound with a composition comprising a FATP5 polypeptide, and determining whether the test compound binds to the FATP5 polypeptide; followed by administering the compound identified as binding to the FATP5 polypeptide to a mammal, and determining whether the test compound modulates dyslipidemia. Thus, identifying a test compound is identified as a candidate compound useful for modulating an FATP5 mediated disorder when the compound modulates dyslipidemia. As used herein, dyslipidemia can include, e.g., elevated serum triglyceride levels, elevated free fatty acid levels, altered plasma lipid composition. In certain embodiments, the composition comprising the FATP5 polypeptide can be a membrane preparation, a cell expressing an FATP5 polypeptide, a tissue expressing an FATP5 polypeptide, or an isolated FATP5 polypeptide. In additional embodiments, the dyslipidemia can be determined by measuring any one or more of: fatty acid/bile acid uptake, free fatty acids, serum triglycerides, and/or plasma lipid composition.

In another embodiment the invention provides a method for identifying a candidate compound useful for dyslipidemia comprising combining a test compound with a composition comprising a cell capable of expressing an FATP5 polypeptide; measuring expression of the FATP5 polypeptide in the composition in the presence and absence of the test compound; then identifying the test compound as a candidate compound for use in modulating dyslipidemia when the FATP5 polypeptide expression in the presence of the compound differs from the FATP5 polypeptide in the absence of the compound. Dyslipidemia can include e.g., elevated serum triglyceride levels, elevated free fatty acid levels, and altered serum lipid composition. In certain embodiments, FATP5 expression can be measured by monitoring one or more of the transcript levels, protein levels, fatty acid or bile acid uptake, or acyl CoA ligase activity.

Still another embodiment provides a method for identifying a candidate compound capable of modulating dyslipidemia comprising combining a test compound and a fatty/bile acid with a cell expressing a FATP5 polypeptide, and measuring uptake of fatty/bile acid in the test cell; then identifying a test compound as a candidate compound for use in modulating dyslipidemia when uptake of fatty/bile acid in the cell in the presence of the compound differs from the uptake of the fatty/bile acid in the absence of compound. Dyslipidemia can include, e.g., elevated serum triglyceride levels, elevated free fatty acid levels, and altered serum lipid composition.

In one embodiment the invention provides a method of identifying whether a compound is a candidate compound capable of modulating an FATP5-mediated cardiovascular disorder, the method comprising, combining a test compound with a composition comprising a FATP5 polypeptide; measuring an acyl-CoA ligase/bile acid CoA ligase activity of the FATP5 polypeptide in the composition in the presence and absence of the test compound; and identifying the test compound as the candidate compound for use in modulating the FATP5-mediated cardiovascular disorder when the FATP5 polypeptide acyl-CoA/bile acid CoA ligase activity in the presence of the compound differs from the FATP5 polypeptide acyl-CoA/bile acid CoA ligase activity in the absence of the compound. As used herein, an FATP5-mediated cardiovascular disorder can be any one of coronary heart disease, atherosclerosis, hypertension,and ischemic heart disease. In certain embodiments, the composition comprising the FATP5 polypeptide can be a membrane preparation, a cell expressing an FATP5 polypeptide, or an isolated FATP5 polypeptide.

Another embodiment includes the above method and further includes administering a test compound which has been identified in the described method as modulating FATP5 acyl CoA/bile acid CoA ligase activity to a mammal and determining whether the test compound modulated an FATP5 mediated process; then identifying the test compound which modulates an FATP5 mediated process as a candidate compound useful for modulating an FATP5 mediated cardiovascular disorder. In certain embodiments, the FATP5 mediated process monitored is selected from one or more of insulin resistance, glucose uptake, fatty acid or bile acid uptake, dyslipidernia, plasma lipid composition, and/or bile acid composition in bile, feces, urine, or plasma.

In another embodiment, the invention provides a method for identifying a candidate compound useful for modulating an FATP5 mediated cardiovascular disorder comprising, combining a test compound with a composition comprising a FATP5 polypeptide, and determining whether the test compound binds to the FATP5 polypeptide; followed by administering the compound identified as binding to the FATP5 polypeptide to a mammal, and determining whether the test compound modulates an FATP5 mediated process. Thus, identifying a test compound is identified as a candidate compound useful for modulating an FATP5 mediated disorder when the compound modulated an FATP5 mediated process. As used herein, an FATP5-mediated cardiovascular disorder can be any one of coronary heart disease, atherosclerosis, hypertension, and ischemic heart disease. In certain embodiments, the composition comprising the FATP5 polypeptide can be a membrane preparation, a cell expressing an FATP5 polypeptide, a tissue expressing an FATP5 polypeptide, or an isolated FATP5 polypeptide. In additional embodiments, the FATP5 mediated process can be selected from any one or more of: fatty acid uptake, bile acid uptake, dyslipidemia, and/or plasma lipid composition.

In another embodiment the invention provides a method for identifying a candidate compound useful for modulating an FATP5 mediated cardiovascular disorder comprising combining a test compound with a composition comprising a cell capable of expressing an FATP5 polypeptide; measuring expression of the FATP5 polypeptide in the composition in the presence and absence of the test compound; then identifying the test compound as a candidate compound for use in modulating an FATP5 mediated cardiovascular disorder when the FATP5 polypeptide expression in the presence of the compound differs from the FATP5 polypeptide in the absence of the compound. The FATP5 mediated cardiovascular disorder can be any of coronary heart disease, atherosclerosis, hypertension, and ischemic heart disease). In certain embodiments, FATP5 expression can be measured by monitoring one or more of the transcript levels, protein levels, fatty acid or bile acid uptake, or acyl CoA ligase or bile acid CoA ligase activity.

Still another embodiment provides a method for identifying a candidate compound capable of modulating an FATP5 mediated cardiovascular disorder comprising combining a test compound and a fatty/bile acid with a cell expressing a FATP5 polypeptide, and measuring uptake of fatty/bile acid in the test cell; then identifying a test compound as a candidate compound for use in modulating an FATP5 mediated cardiovascular disorder when uptake of fatty/bile acid in the cell in the presence of the compound differs from the uptake of the fatty/bile acid in the absence of compound. The FATP5 mediated cardiovascular disorder can be any of coronary heart disease, atherosclerosis, hypertension, ischemic heart disease.

The findings described herein demonstrate that inhibition of FATP5 activity leads to a reduction of insulin resistance, a characteristic of Type 2 diabetes mellitus, and therefore an agents that inhibits FATP5-mediated fatty acid or bile acid uptake are useful in reducing insulin resistance. Still further, FATP5 deficient animals display a beneficial plasma lipid profile, suggesting that agents that inhibit FATP5-mediated fatty acid or bile acid uptake are useful in improving plama lipid profiles. Thus, the present invention provides methods for using compounds capable of modulating FATP5 associated disorders, e.g., metabolic disorders, (e.g., obesity, insulin resistance, type II diabetes, dislipidemia), cardiovascular disorders (e.g., free fatty acid levels, triglyceride levels, coronary artery disease, hypertension, and stroke), and fatty liver disease.

In one embodiment the invention provides for a method of treating a FATP5 mediated metabolic disorder, in an individual comprising administering to the individual an effective amount of an agent that inhibits FATP5 activity. In certain embodiments, the FATP5 mediated metabolic disorder is obesity, insulin resistance, (e.g., type 2 diabetes), dyslipidemia (e.g., elevated serum triglyceride levels, elevated free fatty acid levels), fatty liver disease, and cardiovascular disease (e.g., coronary heart disease, atherosclerosis, hypertension, ischemic heart disease).

In yet another embodiment the invention provides for a genetically engineered nonhuman mammal in which the FATP5 gene has been inactivated

In one embodiment, the genetically engineered transgenic animal is a mouse or a rat.

Additional features and advantages of the invention will be apparent from the following description, exemplification, and claims.

The present invention is based, at least in part, on the discovery that transgenic animals deficient for FATP5 are protected from diet induced obesity and insulin resistance. Still further, FATP5 deficient animals display a beneficial plasma lipid profile. Thus, the present invention provides methods for identifying compounds capable of modulating FATP5 associated disorders, e.g., metabolic disorders, (e.g., obesity, insulin resistance, type 2 diabetes, dyslipidemia), cardiovascular disorders (e.g., free fatty acid levels, triglyceride levels, coronary artery disease, hypertension, and stroke), and fatty liver disease (e.g., steatosis). Methods provided include assaying the ability of a compound to modulate FATP5 nucleic acid expression or FATP5 polypeptide activity, including enzyme activity and fatty acid/bile acid uptake activity. Additional methods provided include modulating an FATP5 mediated cellular process or disorder byway of utilizing compounds identified using the present methods.

The structures and coding sequences for a number of FATP5 genes have been reported. See, e.g., Schaffer, J E et. al Cell. 79: 427-436 (1994); Berger et al., Biochem Biophys Res Commun 247: 255-260 (1998); Hirsch, D et al., Proc Natl Acad Sci 95: 8625-8629 (1998); and PCT International Publication Nos: WO01/021795 and WO99/036537. The mouse and human sequences are reported in GenBank with the Accession Numbers NM_{—}012254, and NM_{—}009512, respectively, and the gene and protein sequences are depicted in SEQ ID NO: 1, and SEQ ID NO: 2 as well as SEQ ID NO: 3, and SEQ ID NO: 4 respectively.

FATP5 is one of a family of proteins which have been identified as fatty acid transporters and acyl CoA synthetases. See, e.g., Schaffer, J E et. al Cell. 79: 427-436 (1994); Berger et al., Biochem Biophys Res Commun 247: 255-260 (1998); Hirsch, D et al., Proc Natl Acad Sci 95: 8625-8629 (1998); PCT International Publication Nos: WO01/021795 and WO99/036537; Steinberg et al., Mol Genet Metab. 58: 32-42 (1999). FATP5 was found to be specifically expressed in liver tissues, and specifically located in hepatocytes, as detected by northern analysis. See, PCT International Publication WO01/021795. Still further, FATP5 has been found to be a bile acid CoA ligase and involved in bile acid recycling. See, e.g., Steinberg et al., J Biol Chem 275: 15605-15608 (2000) and Mihalik et al., J Biol Chem. 277: 28765-28773 (2002). The mouse gene and protein sequences are depicted in SEQ ID NO: 1, and SEQ ID NO: 2 respectively, and the human gene and protein sequences are depicted in SEQ ID NO: 3, and SEQ ID NO: 4 respectively. Our present findings demonstrate surprisingly that the activity of the liver specific FATP5 fatty acid/bile acid CoA ligase is involved in mediating processes involved in metabolic disorders including obesity, insulin resistance, type 2 diabetes and cardiovascular disorders involving aberrant serum lipid profiles.

As used herein, the term “gene” refers to DNA sequences that encode the genetic information (e.g., nucleic acid sequence) required for the synthesis of a single protein (e.g., polypeptide chain). In addition to the “coding sequence”, the sequence that directly codes the amino acid sequence, a gene also includes essential non-coding elements, e.g., promoters, enhancers, silencers, and non-essential flanking and intron sequences. The term “FATP5 gene” refers to a particular mammalian gene that comprises a DNA sequence that encodes the FATP5 protein. Methods provided in the present invention make use of known FATP5 sequences which have been described previously. See, e.g., Genbank Accession refs: NM_{—}012254, and NM_{—}009512. As is understood by one of skill in the art, a gene sequence can contain “sites” (sequence positions) that are different among individuals in a population. Thus, a gene allows for variation of the sequence. Each variant sequence is referred to as an “allele” of the gene. Typically, a particular sequence, usually one that encodes a functional protein, is taken to be a reference or “wild-type” sequence; the term “wild-type” is a descriptive term meant to connote a reference allele, typically an allele that encodes a functional protein or an allele present in a healthy individual. Alleles that differ from the wild-type sequence are referred to as “allelic variants”. Homologous chromosomes are chromosomes that pair during meiosis and contain substantially identical loci. The term “locus” connotes the site (e.g., location) of a gene on a chromosome.

Transgenic FATP5 Animals

The present invention provides a “knockout” or transgenic non-human mammal, e.g., a non-human primate, a rodent such as a mouse or rat, a sheep, a dog, a cow, a pig, a rabbit or a goat. As used herein, “knockout” refers to a genetically modified organism that has a genome in which a particular gene has been disrupted or deleted such that expression of the gene is eliminated or occurs at a reduced level. In particular embodiments, the knockout non-human mammal is a rodent, e.g., a mouse or a rat. For example, a FATP5 knockout mammal comprises disruption of an endogenous FATP5 gene, such that the mammal lacks or has reduced levels of functional FATP5 protein. In a particular embodiment, the non-human knockout mammal is a mouse that lacks a functional FATP5 gene product or exhibits a reduced level of the FATP5 gene product. The transgenic mouse is referred to herein as a “transgenic FATP5 knockout mouse” or a “FATP5 knockout mouse”. In a particular embodiment, the genome of the FATP5 knockout mouse comprises at least one non-functional allele for the endogenous FATP5 gene. Thus, the invention provides a source of cells (e.g., tissue, cells, cellular extracts, organelles) and animals useful for elucidating the function of FATP5 in intact animals whose genomes comprise a functional and referential, sometimes referred to as a “wild type”, FATP5 allele. Any suitable mammal can be used to produce the FATP5 knockout mammal described herein. For example, a suitable mammal can be a non-human primate, a sheep, a dog, a cow, a goat, a mouse (mice), a rat, a rabbit or a pig.

We describe production of mice that lack functional FATP5, methods of using such mice, methods of using cells derived from such mice, methods of using cells lacking or partially lacking FATP5 activity, and in vitro methods for identifying or evaluating agents that modulate FATP5 activity.

As used herein the terms “disruption”, “functional inactivation”, “alteration” and “defect” connote a partial or complete reduction in the expression and/or function of the FATP5 polypeptide encoded by the endogenous gene of a single type of cell, selected cells or all of the cells of a FATP5 knockout mouse. Thus, according to the instant invention the expression or function of the FATP5 gene product can be completely or partially disrupted or reduced (e.g., by 50%, 75%, 80%, 90%, 95% or more) in a selected group of cells (e.g., a tissue or organ) or in the entire animal. As used herein the term “a functionally disrupted FATP5 gene” includes a modified FATP5 gene that either fails to express any polypeptide product or that expresses a truncated protein having less than the entire amino acid polypeptide chain of a wild-type protein and is non-functional (partially or completely non-functional).

Disruption of the FATP5 gene can be accomplished by a variety of methods known to those of skill in the art. For example, gene targeting using homologous recombination, mutagenesis (e.g., point mutation), RNA interference and antisense technology can be used to disrupt a FATP5 gene.

More specifically, the invention provides a knockout mammal, e.g. mouse, whose genome comprises either a homozygous or heterozygous disruption of its FATP gene. A knockout mammal whose genome comprises a homozygous disruption is characterized by somatic and germ cells that contain two nonfunctional (disrupted) alleles of the FATP5 gene, while a knockout mammal whose genome comprises a heterozygous disruption is characterized by somatic and germ cells that contain one wild type allele and one nonfunctional allele of the FATP5 gene.

As used herein, the term “genotype” refers to the genetic makeup of an animal. A particular genotype refers to one or more specific genes, e.g., FATP5. More specifically the term genotype refers to the status of the animal's FATP5 alleles, which can either be intact and functional (e.g., wild-type or +/+); or disrupted (e.g., knockout) in a manner that confers either a heterozygous (e.g., ±); or homozygous (−/−) knockout genotype.

The present invention also provides methods of producing a non-human mammal that lacks a functional FATP5 gene. Briefly, the standard methodology for producing a knockout embryo requires introducing a targeting construct, which is designed to integrate by homologous recombination with the endogenous nucleic acid sequence of the targeted gene, into a suitable embryonic stem cell (ES). The ES cells are then cultured under conditions that allow for homologous recombination (i.e., of the recombinant nucleic acid sequence of the targeting construct and the genomic nucleic acid sequence of the host cell chromosome). Genetically engineered stem cells that are identified as comprising a knockout genotype that comprises the recombinant allele are introduced into an animal, or parent thereof, at an embryonic stage using standard techniques that are well known in the art (e.g., by microinjecting the genetically engineered embryonic stem (ES) cell into a blastocyst). The resulting chimeric blastocyst is then placed within the uterus of a pseudopregnant foster mother for the development into viable pups. The resulting viable pups include potentially chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome, which comprises the disrupted target gene, to be transmitted to the progeny of these founder animals, thereby facilitating the production of “knockout animals” whose genomes comprise a gene that has been genetically engineered to comprise a particular defect in a target gene.

One of skill in the art will easily recognize that the FATP5 gene can be disrupted in a number of different ways, any one of which may be used to produce the FATP5 knockout mammals of the present invention. For example, a knockout mouse according to the instant invention can be produced by the method of gene targeting. As used herein the term “gene targeting” refers to a type of homologous recombination that occurs as a consequence of the introduction of a targeting construct (e.g., vector) into a mammalian cell (e.g., an ES cell) that is designed to locate and recombine with a corresponding portion of the nucleic acid sequence of the genomic locus targeted for alteration (e.g., disruption) thereby introducing an exogenous recombinant nucleic acid sequence capable of conferring a planned alteration to the endogenous gene.

Thus, homologous recombination is a process (e.g., method) by which a particular DNA sequence can by replaced by an exogenous genetically engineered sequence. More specifically, regions of the targeting vector that have been genetically engineered to be homologous or complementary to the endogenous nucleotide sequence of the gene that is targeted for transgenic disruption line up or recombine with each other such that the nucleotide sequence of the targeting vector is incorporated into (e.g., integrates with) the corresponding position of the endogenous gene.

One embodiment of the present invention provides a vector construct (e.g., a FATP5 targeting vector or FATP5 targeting construct) designed to disrupt the function of a wild-type (endogenous) FATP5 gene. In general terms, an effective FATP5 targeting vector comprises a recombinant sequence that is effective for homologous recombination with an endogenous FATP5 gene. For example, a replacement targeting vector comprising a genomic nucleotide sequence that is homologous to the target sequence operably linked to a second nucleotide sequence that encodes a selectable marker gene exemplifies an effective targeting vector. Integration of the targeting sequence into the chromosomal DNA of the host cell (e.g., embryonic stem cell) as a result of homologous recombination introduces an intentional disruption, defect or alteration (e.g., insertion, deletion or substitution) into the targeted sequence of the endogenous gene, e.g., the FATP5 gene. One aspect of the present invention is to replace all or part of the nucleotide sequence of a non-human mammalian gene that encodes the FATP5 polypeptide, thereby making a transgenic FATP5 knockout.

One of skill in the art will recognize that any FATP5 genomic nucleotide sequence of appropriate length and composition to facilitate homologous recombination at a specific site that has been preselected for disruption can be employed to construct a FATP5 targeting vector. Guidelines for the selection and use of sequences are described for example in Deng, C. and Cappecchi, M., 1992, Mol. Cell. Biol., 12:3365-3371, and Bollag, R. et al., 1989, Annu. Rev. Genet., 23:199-225. For example, a wild-type FATP5 gene can be mutated and/or disrupted by inserting a recombinant nucleic acid sequence (e.g., a FATP5 targeting construct or vector) into all or a portion of the FATP5 gene locus. For example, a targeting construct can be designed to recombine with a particular portion within the enhancer, promoter, coding region, start codon, noncoding sequence, introns or exons of the FATP5 gene. Alternatively, a targeting construct can comprise a recombinant nucleic acid that is designed to introduce a stop codon after an exon of the FATP5 gene.

Suitable targeting constructs of the invention can be prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y.; which disclosures are hereby incorporated by reference. Appropriate vectors include a replacement vector such as the insertion vector described by Capecchi, M., 1989, 30 Science, 244:1288-92, which disclosure is hereby incorporated by reference; or a vector based on a promoter trap strategy or a polyadenylation trap, or “tag-and-exchange” strategy described by Bradley, et al., 1992, Biotechnology (NY), 10:534-539; and Askew, G. et al., 1993, Mol. Cell. Biol., 13:4115-4124, which disclosures are also incorporated herein by reference.

One of skill in the art will readily recognize that a large number of appropriate vectors known in the art can be used as the basis of a suitable targeting vector. In practice, any vector that is capable of accommodating the recombinant nucleic acid sequence required to direct homologous recombination and to disrupt the target gene can be used. For example, pBR322, pACY164, pKK223-3, pUC8, pKG, pUC19, pLG339, pR290, pKC101 or other plasmid vectors can be used. Alternatively, a viral vector such as the lambda gt11 vector system can provide the backbone (e.g. cassette) for the targeting construct.

According to techniques well known to those of skill in the art genetically engineered (e.g., transfected using electroporation or transformed by infection) embryonic stem cells are routinely employed for the production of transgenic and knockout non-human embryos. Embryonic stem (ES) cells are pluripotent cells isolated from the inner cell mass of a mammalian blastocyst. ES cells can be cultured in vitro under appropriate culture conditions in an undifferentiated state and retain the ability to resume normal in vivo development and differentiation as a result of being combined with a blastocyst and introduced into the uterus of a pseudopregnant foster mother.

Those of skill in the art will recognize that various stem cells are known in the art, for example AB-1, HM-1, D3. CC1.2, E-14T62a, RW4 or JI (Teratomacarcinoma and Embryonic Stem Cells: A Practical Approach, E. J. Roberston, ed., IRL Press).

It is to be understood that the FATP5 knockout mammals described herein can be produced by methods other than the embryonic stem cell method described above, for example, by the pronuclear injection of recombinant genes into the pronuclei of one-cell or transformed embryos or other gene targeting methods that do not rely on the use of a transfected or transformed ES cell, and that the exemplification of the single method outlined above is not intended to limit the scope of the invention to animals produced solely by this protocol.

The FATP5 knockout mammals described herein can also be bred (e.g., inbred, outbred or crossbred) with appropriate mates to produce colonies of animals whose genomes comprise at least one non-functional allele of the endogenous gene that naturally encodes and expresses functional FATP5. Examples of such breeding strategies include but are not limited to: crossing of heterozygous knockout animals to produce homozygous animals; outbreeding of founder animals (e.g., heterozygous or homozygous knockouts) with a mouse whose inbred genetic background confers aberrant insulin and/or glucose homeostasis or that provides an animal model of diabetes and crossbreeding a founder animal with an independent transgenic animal that has been genetically engineered to overexpress a gene associated with increased susceptibility to diabetes and/or obesity. For example, a founder knockout mouse could be bred with an ob/ob mouse, a db/db mouse and/or an AY mouse.

The FATP5 knockout mammals, e.g., mice, are useful for a variety of purposes. In one embodiment, a FATP5 knockout cell or cell line can be engineered using skills known in the art. For example, cells that do not possess an endogenous FATP5 gene or that normally do not express FATP5 can be engineered to do so. For example, an exogenous FATP5 gene can be introduced into a cell that does not possess an endogenous FATP5 gene wherein the cell expresses FATP5 due to the presence of the exogenous FATP5 gene. Alternatively, exogenous nucleic acid can be spliced into the genome of a cell that does not normally express FATP5 in order to “turn on” the normally silent FATP5 gene. The agent can be for example, a nucleic acid molecule, a polypeptide, an organic molecule, an inorganic molecule, a fusion protein etc.

Subsequently the FATP5 gene in the engineered cells can be disrupted using the methods described herein and known to those of skill in the art for use in the methods and compositions of the present invention.

As a result of the disruption of the FATP5 gene, the FATP5 knockout mouse of the present invention can manifest a particular phenotype. The term phenotype refers to the resulting biochemical or physiological consequences attributed to a particular genotype. In the situation where a knockout mouse has been created, the phenotype observed is a result of the loss of the gene that has been knocked out. In one embodiment, the FATP5 knockout mouse has altered insulin/glucose homeostasis and responsiveness to diet induced insulin resistance. The FATP5 knockout mouse exhibits reduced insulin resistance when compared to a wild type mouse fed a high-fat diet. In addition, FATP5 knockout mice exhibit reduced serum triglyceride and free fatty acid levels. Furthermore the FATP5 knockout mice exhibit a decrease in adiposity. Specifically, the FATP5 knockout mice display a significant decrease in white fat mass when compared to wild type control mice. This phenotype is exacerbated when the animals are fed a high-fat diet. Additionally the FATP5 mice displayed a decrease in food intake and an increase in energy expenditure.

The present invention is based on the discovery in FATP5 knockout mice, that insulin resistance is reduced. The reduction in insulin resistance allows for a higher level of glucose metabolism. As Type 2 diabetes mellitus is characterized by insulin resistance and a low level of glucose metabolism, the results described herein allow for methods of treating Type 2 diabetes mellitus as well as associated phenotypes such as, for example, decreased glucose metabolism, insulin resistance and fatty acid accumulation.

In another embodiment the findings described herein also demonstrate that inhibition of FATP5 activity results in a beneficial plasma lipid profile. In a further embodiment the present invention is also based on the recognition that FATP5 knockout mice have reduced levels of serum triglycerides and free fatty acids. Therefore, agents that inhibit FATP5-mediated fatty acid transport are useful in treating cardiovascular diseases in which elevated lipid and/or free fatty acids are a factor of the cardiovascular disease. Cardiovascular diseases that are associated with increased lipid and/or free fatty acids includes coronary heart disease, atherosclerosis, hypertension, and ischemic heart disease.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, arterial inflammation, coronary microembolism, tachycardia, bradycardia, pressure overload, vascular heart disease, congestive heart failure, angina, heart failure, hypertension, myocardial infarction, coronary artery disease, coronary artery spasm, ischemic disease, and arrhythmia.

In another aspect, the findings described herein also indicate that inhibiting FATP5 activity has a beneficial effect on adiposity. In yet another aspect the invention provides for inhibition of FATP5 activity, resulting in increased energy expenditure. This invention is based upon the fact that FATP5 knockout mice display a significant decrease in white fat mass when compared to wild type mice. Furthermore, when the mice are fed a high-fat diet the decreased in adiposity of the FATP5 knockout mice becomes more obvious. In a further embodiment FATP5 knockout mice are completely protected from high-fat-induced increases in body weight and adiposity. In another embodiment the FATP5 knockout animals displayed a decrease in food uptake when compared to wild type controls. Additionally, FATP5 knockout animals displayed increased expenditure of energy as compared to wild type control animals.

Assays

The present invention provides methods for identifying a compound capable of treating a metabolic disorder, e.g., body weight, obesity, insulin resistance, type 2 diabetes, dyslipidemia, e.g., elevated serum triglyceride levels, elevated free fatty acid levels, fatty liver disease and cardiovascular disease, e.g., coronary heart disease, atherosclerosis, hypertension. The methods include assaying the ability of the compound to modulate FATP5 nucleic acid expression or FATP5 acyl-CoA ligase activity. In certain aspects, the ability of the compound to modulate FATP5 acyl-CoA ligase activity can be determined by detecting the production of phosphate, acyl-CoA, or AMP. In additional aspects, the ability of the compound to modulate FATP5 acyl-CoA activity can be determined by detecting consumption of coenzyme A, ATP, or fatty acid/bile acid.

In still other aspects, the ability of the compound to modulate FATP5 acyl-CoA activity is determined by detecting modulation of FATP mediated processes. Such processes can include, for example, cellular processes (e.g., fatty acid or bile acid uptake), or body processes, (e.g., body weight, body fat composition, energy expenditure or feeding behavior, blood glucose levels, serum triglyceride levels, serum free fatty acid levels, bile acid composition in bile, plasma, urine or feces). In still other embodiments, FATP5 mediated processes can include disorders (e.g., fatty liver disease, cardiovascular disease (e.g., coronary heart disease, atherosclerosis, hypertension, and ischemic heart disease), obesity, insulin resistance, type 2 diabetes, dyslipidemia.

Further aspects of the invention provide a method for the identification of agents (e.g., therapeutic agents) that modulate, e.g., increase or decrease, FATP5 function; and a method of treating diseases or conditions associated with FATP5 function (e.g., obesity, insulin resistance, type 2 diabetes, dyslipidemia, fatty liver disease and cardiovascular disease).

It has now been determined that inhibition of FATP5-mediated uptake of fatty acids or bile acids significantly reduces “insulin resistance”, a reduced biological response to either exogenous or endogenous insulin. Insulin resistance is a characteristic of Type 2 diabetes mellitus and leads to the inability of the affected individual to metabolize glucose, e.g., clear glucose from the blood stream and take up glucose into cells, and to increased production of glucose in the liver.

That FATP5 facilitates uptake of fatty acids and bile acids and has an acyl-CoA ligase activity suggests that the enzymatic and observed transport activities are related. However, acyl-CoA ligase activity can be measured in the absence of transport to further characterize just the enzymatic activity.

Thus, the data presented herein identify a therapeutic target molecule for treating FATP5 disorders, e.g., body weight, obesity, insulin resistance, type 2 diabetes, dislipidemia, free fatty acid levels, triglyceride levels, coronary artery disease, hypertension, and stroke. Inhibition of FATP5 reduces modified fatty and/or bile acid uptake and insulin resistance, and decreases serum triglycerides and free fatty acids. The invention therefore is directed, in part, to methods for identifying an agent that inhibits FATP5 for use in treating obesity, insulin resistance, type 2 diabetes, dyslipidemia, fatty liver disease and cardiovascular disease.

The identification of an agent that modulates or alters an FATP5 activity, e.g., FATP5-mediated fatty acid or bile acid uptake or acyl-CoA ligase activity, involves measuring the FATP5-mediated fatty acid or bile acid uptake and/or FATP5 acyl-CoA ligase activity. Such activity can be measured in vivo, ex vivo and/or in vitro. FATP5-mediated fatty acid or bile acid uptake activity can be measured, for example, by determining the level (used herein to refer to either amount or rate) of acyl-CoA-modified fatty acid or bile acid accumulation in cells; bile acid composition in liver, bile, plasma, urine or feces; serum triglyceride levels; free fatty acid levels; plasma lipid composition; glucose uptake; glucose clearance; or hepatic glucose output. Acyl-CoA ligase activity can be measured, for example, by determining the level of acyl-CoA-modified fatty acid or acyl-CoA modified bile acid accumulation; consumption of coenzyme A, ATP, and/or fatty acid/bile acid; and/or production of phosphate, acyl-CoA or AMP (see Examples ). Suitable methods for determination of acyl CoA ligase activity are known in the art and can be adapted to suit the methods provided in the present invention. See, e.g., Steinberg, S J et al., Biochem Biophys Res Comm. 257: 615-621 (1999); Steinberg, S J et al., Mol Genet Metab. 58: 32-42 (1999); Steinberg, S J et al., J Biol Chem. 275: 15605-15608 (2000).

FATP5-mediated fatty acid or bile-acid uptake can also be determined by monitoring the white fat weight of mice fed a chow diet. As illustrated Example 2, experiments were conducted to measure body weight and adiposity. A group of wild type mice and a group of FATP5 knockout mice were maintained on a standard lab chow diet for six months. FATP5 knockout mice showed a significantly lower white fat mass compared to the wild type controls. Thus FATP5 activity can be assessed in animals by measuring white fat mass.

Similarly FATP5-mediated fatty acid or bile acid uptake can be assessed by monitoring body weight, energy expenditure and/or food intake of mice on a high fat diet. As illustrated in Examples 3 and 4, experiments were conducted to measure body weight and food intake in mice maintained on a high fat diet. The FATP5 knockout mice displayed a significantly lower body weight than wild type mice maintained on the same diet. Additionally the FATP5 knock out mice displayed a significantly lower food intake than the wild type mice. Also, as demonstrated in Example 5, FATP5 knock-out mice had significantly higher energy expenditure than wild-type mice. Thus FATP5 activity can be assessed in mice by determining body weight, energy expenditure and/or food intake in animals maintained on a high fat diet.

FATP5 mediated fatty acid uptake can also be assessed by monitoring total fat mass of mice fed a high fat diet. As illustrated in Example 4, experiments were conducted to measure fat mass in FATP5 knock out mice and wild type mice maintained on a high fat diet. The data indicates that FATP5 knock-out mice had a significantly lower fat mass than wild type mice. Thus, another aspect of the invention provides for assessment of FATP5 activity by determination of total fat mass of animals maintained on a high fat diet.

In another aspect, the FATP5 knock out animals demonstrated a decrease in the level of free fatty acids and serum triglycerides. As illustrated in Example 6, experiments were conducted to measure the levels of free fatty acids and serum triglycerides in FATP5 knock-out and wild type mice maintained on a high fat diet. Mice were then fasted overnight and serum collected. The results indicate that FATP5 knock-out mice had lower serum levels of free fatty acids and triglycerides. Thus, in an additional embodiment, FATP5 activity can be assessed by monitoring the serum levels of free fatty acids and triglycerides of animals maintained on a high fat diet.

In another aspect the FATP5 knock-out animals demonstrate a decrease in the percent wet weight of liver lipids. As illustrated in Example 7, FATP5 knock-out mice and wild type mice were maintained on a high fat diet. The animal's livers were collected and the percent wet weight of liver lipids was measured. FATP5 knock-out animals displayed a signifantly lower percent wet weight of liver lipids compared to wild type control animals. Thus, another aspect for assessment of, FATP5 activity is provided by measuring the liver lipid content of animals maintained on a high fat diet.

In yet another aspect, the phenotype of the FATP5 knock-out animals is manifested as a protection from diet-induced insulin resistance. Example 5 illustrates the results of experiments conducted to compare the rate of glucose disappearance after i.p. injection of glucose. The rate of glucose disappearance reflects the rate of insulin-stimulated whole body glucose uptake and insulin-mediated suppression of hepatic glucose output between wild-type mice and knockout mice. To induce insulin resistance, a group of wild-type mice and a group of FATP5 knockout mice were maintained on a high fat diet. While the ability to clear glucose decreased in wild-type mice fed a high-fat diet, indicating the development of insulin-resistance, FATP5 knock-out mice were completely protected from the effects of lipid infusion and high-fat diet, showing normal levels of glucose disappearance, indicating protection from insulin resistance by FATP5 deletion. Thus another embodiment of the invention provides methods for assessment of FATP5 activity by determining protection from insulin resistance induced by a high fat diet. This can be determined using methods known in the art, including measurement via a glucose tolerance test.

In still another aspect, the invention provides methods for assessment of FATP5 activity by monitoring de novo bile acid biosynthesis or cholesterol biosynthesis, and the genes involved in the biosynthesis processes. It has been found that the FATP5 knock-out animals demonstrate upregulation in the genes involved in cholesterol biosynthesis, bile acid biosynthesis, and downregulation of genes involved in gluconeogenesis. As illustrated in Example 10, FATP5 knock-out mice and wild type mice were monitored for bile acid content, as well as gene expression of biosynthetic pathways. The animal's livers were collected and the bile acids and gene expression of tissues was analyzed. FATP5 knock-out animals displayed a deficiency in conjugation of recirculated bile acids; as well as increased expression of genes involved in de novo bile acid biosynthesis and cholesterol biosynthesis compared to wild type control animals. Additionally, FATP5 knock-out animals displayed diminished expression of genes involved in gluconeogenesis as compared to wild type control animals. Thus, another aspect for assessment of, FATP5 activity is provided by measuring the bile acid conjugation, as well as bile acid or cholesterol biosynthesis pathways.

The acyl-CoA ligase activity of FATP5 can be measured, for example, by determining the level of acyl-CoA-modified fatty acid or bile acid accumulation; consumption of coenzyme A, ATP and fatty acid/bile acid; and/or production of phosphate, acyl-CoA or AMP. Labeled reagents, e.g., fluorescently labeled, enzymatically labeled or radiolabeled, reactants or products can be obtained and detected according to methods known in the art. For example, consumption of coenzyme A, ATP and fatty acids/bile acids can be monitored by monitoring the level of radiolabeled coenzyme A, ATP and fatty acids/bile acids. Alternatively, labeled phosphate, acyl-CoA or AMP can also be detected and monitored.

For example, in one type of assay, e.g., a cell-based assay, determining the effect of a modulating compound (e.g., an inhibitor) on an FATP5 activity includes use of a BODIPY-fatty acid (4,4-difluoro -5-methyl -4-bora -3a,4a-diaza-s-indacene-dodecanoic acid), and a radiolabeled fatty acid or a bile acid (e.g. ¹⁴C-laurate). In this assay, cells expressing FATP5 are exposed to the fatty acid or bile acid, unincorporated fatty/bile acid is removed by washing and cell-associated fluorescence or radioactivity is measured using appropriate detection devices. Any appropriate cells expressing FATP5 can be used in cell based assays, including transfected cell lines (e.g., stably transfected HEK 293 cells expressing FATP5), and cells which naturally express FATP5 (e.g., primary hepatocytes).

In another type of cell-based assay, a change of intracellular pH resulting from the uptake of fatty acids can be followed by an indicator fluorophore. The fluorophore can be taken up by the cells in a preincubation step. Fatty acids are added to the cell medium, and after some period of incubation to allow FATP5-mediated uptake of fatty acids, the change in max of fluorescence can be measured, as an indicator of a change in intracellular pH, as the max of fluorescence of the fluorophore changes with the pH of its environment, thereby indicating uptake of fatty acids. One such fluorophore is BCECF (2′, 7′-bis(2-carboxyethyl)-5(6)-carboxyfluorescein. See, Rink, T. et al., 1982. J. Cell Biol., 95: 189-196. Thus, for this assay, fatty acid uptake is indicated by a decrease in intracellular pH.

Yet another type of cell based assay includes exposing cells to labeled unconjugated bile acid, then monitoring uptake of bile acids be determining the amount of conjugated bile acid in the cell, using appropriate detection methods.

The availability of FATP5 knockout cells and mammals facilitate the genetic dissection of FATP5-mediated signaling pathways and allow for the identification of FATP5 specific inhibitors. For example, an agent that inhibits a function of FATP5 equally in a knockout cell line and its wild-type parental cell line would be recognized as a non-FATP5-specific inhibitor, while an agent that inhibits a FATP5 function in a wild-type cell line and has no effect in the knockout cell line, would be recognized as a FATP5 specific inhibitor.

Other embodiments of the invention provide methods of identifying an agent that inhibits the activity (function) of mammalian FATP5 including, e.g., an antagonist, a partial antagonist. An inhibitor of FATP5 includes any agent that inhibits FATP5 gene expression (either a partial or a complete inhibition of expression) or function (either a partial or a complete inhibition of function) of the FATP5 protein. For example, methods described herein for measuring FATP5 activity can be used in screening methods as well as methods known in the art for measuring FATP5 activity. According to the instant invention, the agent can be combined with a cell, a primary tissue, and/or administered to a whole animal. As demonstrated in the following examples, administration can be accomplished in various ways such as the addition to culture media, tissue perfusion, by expressing it from a vector, or by injection.

In one embodiment, an agent can be identified or evaluated based on its ability to inhibit FATP5, thereby reducing insulin resistance. Such a screen can be performed in vivo or ex vivo using cells isolated from animals which have been treated.

For example, an insulin resistant mouse or cells exhibiting insulin resistance can be used for the screening methods described herein. A baseline level, used herein to refer to a level prior to a particular manipulation of a system, for glucose metabolism (e.g., uptake or clearance), measured by any of the methods described herein or known in the art, can be determined. Upon addition of a test agent, glucose metabolism can again be measured. Under insulin resistant conditions, glucose metabolism is altered. If, in the presence of the test agent, glucose metabolism is reversed toward normal, then insulin resistance has been decreased and the agent is an inhibitor of FATP5.

The screening methods described herein can further comprise the use of any suitable control. For example, in one embodiment, the screening method can further comprise administering to a wild-type mouse an amount of glucose sufficient to stimulate insulin production in the absence and the presence of the agent; and administering to a FATP5 knockout mouse an amount of glucose sufficient to stimulate insulin production in the presence of the agent. The rate of glucose clearance of the mice is determined. The rate of glucose clearance of the wild-type mouse in the presence of the agent is compared to rate of glucose clearance of the wild-type mouse in the absence of the agent; and the rate of glucose clearance of the FATP5 knockout mouse in the presence of the agent is compared to the rate of glucose clearance of the FATP5 knockout mouse in the absence of the agent.

If the rate of glucose clearance of the wild-type mouse in the presence of the agent is increased compared to the rate of glucose clearance by the wild-type mouse in the absence of the agent, and the rate of glucose clearance by the FATP5 knockout mouse in the presence of the agent is similar to the level produced by the knockout mouse in the absence of the agent, then the agent specifically inhibits FATP5. In the screening methods of the present invention, the rate of glucose taken up by the cells, by the wild-type mice or the FATP5 knockout mice can be determined using a variety of methods as described herein or known to those of skill in the art. Methods of screening agents useful for treating body weight, obesity, insulin resistance, type II diabetes, dislipidemia, free fatty acid levels, triglyceride levels, coronary artery disease, hypertension, and stroke, utilizing mammals are also within the scope of the invention. A suitable in vivo or ex vivo screening method for identifying agents useful for treating insulin resistance comprises administering to a mammal, tissue or cultured cell line that comprises a wild-type FATP5 gene, e.g., a mouse, an amount of glucose sufficient to stimulate insulin production and a test agent.

The rate of glucose clearance of the mouse is measured. The rate of glucose clearance of the mouse is compared to the rate of glucose clearance by the same or a similar mouse administered the same amount of glucose in the absence of the test agent. If the rate of glucose clearance of the mouse is increased in the presence of the test agent, then an agent useful in the treatment of insulin resistance has been identified.

Similarly, a suitable in vivo screening method for identifying agents useful for treating non-insulin dependent diabetes mellitus comprises administering to a mammal, e.g., a mouse, which comprises a wild-type FATP5 gene, an amount of glucose sufficient to stimulate insulin production and a test agent. The rate of glucose clearance of the mouse is measured. The rate of glucose clearance of the mouse is compared to the rate of glucose clearance by the same or a similar mouse administered the same amount of glucose in the absence of the test agent. If the rate of glucose clearance of the mouse is increased in the presence of the test agent, then an agent useful in the treatment of non-insulin resistant diabetes mellitus has been identified.

In vivo and ex vivo methods for screening agents useful for modulation of levels of free fatty acids and triglyceride, e.g., serum, are also contemplated. Such methods include administering to a mammal that comprises a wild-type FATP5 gene, e.g., a mouse, a test agent. The amount of triglyceride and/or fatty acids is measured after administration of the test agent and compared to the amount of triglyceride and/or fatty acid prior to administration of the test agent. If the amount of triglycerides and/or fatty acids differ significantly after administration of the test agent, then an agent that modulates triglyceride and/or free fatty acid levels has been identified. Such modulation includes both an increase and a decrease in triglyceride and/or serum triglyceride levels. The production or accumulation of various fatty acid metabolites can also be measured in this manner.

The acyl-CoA ligase activity of FATP5 can be measured, as described above, by in vivo, ex vivo and in vitro methods. Screening methods based on acyl-CoA ligase activity can be, for example, the identification of an agent that binds to and inhibits the in vitro ligase activity of FATP5 as measured, for example, by the accumulation of acyl-CoAs. Additionally, the accumulation of acyl-CoAs in cells (e.g., adipose, muscle or heart cells), tissues or animals can also be used as an assay for screening for an agent that inhibits FATP5 acyl-CoA ligase activity.

In one embodiment, the invention is directed to an in vitro or an in vivo method for identifying an agent useful in treating insulin resistance. As described herein, inhibition of FATP5 leads to a reduction or elimination in insulin resistance. Therefore, an agent that inhibits FATP5 will be useful as an agent that reduces insulin resistance.

The method includes contacting a test agent with a mammalian FATP5 (either in vitro, in vivo, or ex vivo, e.g., in a cultured cell line in the native state or engineered to overexpress mammalian FATP5). The level of acyl-CoA ligase activity of the mammalian FATP5 in the presence of the test agent is determined and compared to the level of acyl-CoA ligase activity of the mammalian FATP5 in the absence of the test agent. When the level of the acyl-CoA ligase activity is decreased in the presence of the test agent, an agent useful for the treatment of insulin resistance has been identified.

Similarly, in another embodiment, the invention is directed to an in vitro or an in vivo method for identifying an agent useful in treating non-insulin dependent diabetes mellitus. Since insulin resistance is characteristic of Type 2 diabetes (non-insulin dependent diabetes mellitus), an agent that reduces insulin resistance, e.g., an agent that inhibits FATP5, is useful for treating non-insulin dependent diabetes mellitus. The method includes contacting a test agent with a mammalian FATP5. The level of acyl-CoA ligase activity of the mammalian FATP5 in the presence of the test agent is determined and compared to the level of acyl-CoA ligase activity of the mammalian FATP5 in the absence of the test agent. When the level of the acyl-CoA ligase activity is decreased in the presence of the test agent, an agent useful for the treatment of non-insulin dependent diabetes mellitus has been identified.

In vitro or an in vivo methods for identifying an agent useful for reducing triglyceride and/or fatty acid levels are also contemplated. The methods include contacting a test agent with a mammalian FATP5. The level of acyl-CoA ligase activity of the mammalian FATP5 in the presence of the test agent is determined and compared to the level of acyl-CoA ligase activity of the mammalian FATP5 in the absence of the test agent. When the level of the acyl-CoA ligase activity differs in the presence of the test agent, an agent useful for the treatment of triglyceride and/or fatty acid accumulation has been identified.

In particular embodiments, the levels of acyl-CoA ligase activity are increased in the presence of the agent. In alternative embodiments, the levels of acyl-CoA ligase are decreased in the presence of the agent. The altered activity, i.e., modulation of activity, can be measured by the methods described herein.

Therapeutic Methods

The present invention also relates to methods of treatment or prevention of conditions (e.g., hyperglycemia) or diseases or disorders (e.g., Type 2 diabetes) affected by FATP5 function. For example the invention provides a method of treating (e.g., alleviating the symptoms of or preventing (e.g., in a individual who is predisposed to develop) altered glucose/insulin homeostasis. The term “individual” as used herein is intended to encompass any single member of a species including a human subject. In one embodiment, the invention provides a method of increasing an individual's whole body glucose clearance by administering to the individual an agent that inhibits FATP5 activity.

The increase can result from an increase of insulin-stimulated glucose uptake in muscle and adipose cells or from a decrease in insulin-meadiated suppression of glucose production in liver cells. In certain embodiments, the invention also provides a method of decreasing the accumulation of serum triglycerides and free fatty acids.

In another embodiment, the invention provides a method of decreasing blood glucose in an individual comprising administering to the individual an agent that inhibits FATP5 activity. The invention further provides a method of treating diabetes (e.g., Type 2 diabetes) in an individual comprising administering to the individual an agent that inhibits FATP5 activity.

The agent for use in the methods of the present invention can be for example, a nucleic acid molecule (e.g., DNA, RNA, antisense DNA, antisense RNA), a protein, a peptide, a polypeptide, a glycoprotein, a polysaccharide, an organic molecule, an inorganic molecule, a fusion protein, etc.

The agents (e.g., therapeutic agents such as FATP5 inhibitors) can be administered to a host in a variety of ways. Potential routes of administration include intradermal, transdermal (e.g., utilizing slow release polymers), intramuscular, intraperitoneal, intravenous, subcutaneous or oral routes. Any convenient route of administration can be used, for example, infusion or bolus injection, or absorption through epithelial or mucocutaneous linings. The agent can be administered in combination with other components such as pharmaceutically acceptable excipients, carriers, vehicles or diluents.

In the treatment methods designed to inhibit the function of FATP5, an “effective amount” of the agent is administered to an individual. As used herein, the term “effective amount”, e.g., an amount that inhibits (or reduces) the activity of FATP5, and results in a significant, e.g., a statistically significant difference, e.g., increase, decrease, in a cellular function that is normally subject to regulation, e.g., negative regulation by FATP5. For example, an effective amount of a therapeutic agent administered to an individual who is hyperglycemic would comprise an amount sufficient to alter (inhibit) FATP5 facilitated uptake of fatty acids or bile acids that thereby facilitates the effective use of insulin, i.e., reduces insulin resistance. The amount of agent required to inhibit FATP5 activity will vary depending on a variety of factors including the size, age, body weight, general health, sex and diet of the host as well as the time of administration, and the duration or stage of the particular condition or disease that is being treated. Effective dose ranges can be extrapolated from dose-response curves derived in vitro or an in vivo test system that utilizes, for example, the non-human FATP5 knockout mice described herein.

All patents, patent applications and references referred to herein are incorporated by reference in their entireties.

The following examples are offered for the purpose of illustrating the present invention and are not to be construed to limit the scope of this invention.

### Exemplification

### EXAMPLE 1

### Generation of a FATP5 Knock-Out Mice

Generation of FATP5 Targeting Constructs:

Genomic DNA containing the mouse FATP5 locus was obtained by screening a 129/Sv genomic bacterial artificial chromosome library (Research Genetics) with a fragment containing the 5′ end of the mouse FATP5 coding sequence. Positive clones were digested with PstI. FATP5-containing fragments were identified by Southern blotting and subcloned into a standard shuttle vector. To generate a targeting construct, PCR primers were designed that allowed amplification of genomic DNA just upstream of the initiation codon (5′ arm) and genomic DNA just downstream of the first coding exon (3′ arm). PCR primers were designed to introduce an XbaI site at the 3′ end of the 5′ arm and a PstI site at the 5′ end of the 3′ arm. Amplified arms were subcloned into the targeting vector pGEMneo-lacZ (Promega). See Mercer et al., Neuron 7:703-716, 1991. The lacZ coding sequence was located immediately downstream of the 5′ arm, thus putting lacZ under the transcriptional control of the FATP5 promoter.

To allow confirmation of recombination events by Southern Blot, probes were generated from genomic DNA immediately adjacent to the arms present in the targeting construct. To generate the 5′ probe, a fragment of genomic DNA starting immediately upstream of the 5′ arm was amplified by PCR and subcloned into a shuttle vector. To generate a 3′ probe, a fragment of genomic DNA starting 90 nucleotides downstream of the 3′ arm was amplified by PCR and subcloned into a shuttle vector. Correct recombination event results in deletion of the first amino acids of the FATP5 protein and replaces the first coding exon of the FATP5 gene with the lacZ gene, followed by the PGK-neo cassette.

Generation of Targeted ES Cells:

The 129SvEv ES cell line was cultured on SNL76/7 mitotically inactive feeder cells as described previously. See Robertson, E. J., ed. Teratocarcinomas and Embryonic Stem Cells: A practical approach, IRL, Oxford, 1987. Pp71-112. Electroporation of the cells was performed as described previously. See, Huszar et al., (1997) Cell 88:131-141. Briefly, cells were trypsinized and resuspended at a concentration of 1.0×107/ml in PBS (Ca and Mg-free, Gibco). A 0.7 ml aliquot (7×106 cells) was mixed with 20 μg of the linearized targeting vector and pulsed at 250V, 500 μF (Bio-Rad Gene Pulser). The cells were then diluted in culture medium, plated at 1-2×106 per 100 mm plate containing feeder cells and placed under selection 24 h later in G418 sulfate (300 μ/ml solution; Gibco). G418-resistant clones were picked, dissociated with trypsin, and divided into one well each of two 96-well plates. Upon confluence, ES cells were frozen in one of the 96-well plates as described previously. See, Ramirez-Solis et al (1993) Meth Enzymol 225:855-878. DNA was prepared from the other plate for screening by Southern blot hybridization. Positive clones were extended and analyzed by Southern Blot hybridization using 5′ and 3′ flanking probes to confirm recombination.

Generation of FATP5-Deficient Mice:

ES cell clones that had undergone correct homologous recombination events were injected into blastocysts and then transferred to pseudopregnant female mice to generate chimeric offspring. Male chimeras were mated with C57B1/6J females to obtain germline transmission of the disrupted FATP5 gene. Resulting heterozygotes were interbred to generate mice homozygous (FATP knockout, FATPKO) or heterozygous for the FATP5 mutation, along with wild-type litter mates. For some experiments, homozygous FATP5KO mice generated by interbreeding FATP5 knockout mice for one generation, and wild-type litter-mates generated by interbreeding wild-type littermates for one generation, were used.

### EXAMPLE 2

### Body Weight and Adiposity of Wild-Type and FATP5 Knock Out Mice Maintained on a Chow Diet

Six male FATP5 KO and wild-type littermates (control) were maintained on a standard chow diet (20 kcal % fat) from birth until 26 weeks old. Body weight, white fat, e.g., epididymal fat pad plus retroperitoneal fat pad, and brown fat, e.g., intrascapular brown adipose tissue, weight was measured at the end of the 26-week period. FATP5 KO mice displayed a significantly decreased white fat mass when compared to wild type controls. See Table I.

TABLE I
Effect of FATP5 deletion on Body Weight and Adiposity on a chow diet

| | Body Weight | White Fat | Brown Fat | Liver Weight |
|---|---|---|---|---|
| Genotype | (g) | Weight (g) | Weight (g) | (g) |
| Wild-type | 36.87 ± 2.05 | 2.25 + 0.2 | 0.189 + 0.033 | 1.39 + 0.13 |
| FATP5 -/- | 34.57 + 2.59 | 1.53 + 0.24* | 0.133 + 0.013 | 1.62 + 0.14 |

*p < 0.05

### EXAMPLE 3

### Body Weight/Food Intake of Wild-Type and FATP5 Knock Out Mice Maintained on a High Fat Diet

Fourteen male FATP5 KO and wild type mice were maintained on a high fat diet (58 kcal % fat) beginning at 8 weeks of age. Body weights were measured before beginning the high-fat diet and again after 11 weeks on the high fat diet. Similarly, cumulative food intake was measured during the 11 week period. FATP5 KO mice were completely protected from high-fat-induced increases in body weight when compared to wild type controls. Additionally, the FATP5 mice maintained on the high fat diet displayed decreased food intake. See Table II.

TABLE II
Effect of FATP5 deletion on Body Weight/Food
Intake during High Fat Diet

| | Starting Body | | Body Weight | Food Intake |
|---|---|---|---|---|
| Genotype | Weight (g) | Diet | 11 weeks (g) | 11 weeks (kcal) |
| Wild-type | 30.27 ± 0.77 | High Fat | 40.84 ± 1.48 | 1030.66 ± 23.86 |
| FATP5 -/- | 29.59 ± 0.66 | High Fat | 36.7 ± 0.9 | 946.57 ± 28.84 |
| Wild-type | 32.59 ± 0.75 | Chow | 36.62 ± 0.77 | nd |

*p < 0.05

### EXAMPLE 4

### Body Composition in FATP5 Knock Out and Wild Time Mice Maintained on a High Fat Diet

Fourteen male FATP5 KO and wild type mice were maintained on a high fat diet as described in Example 3. FATP5 KO mice were completely protected from high-fat-induced increases in body weight, as described above. Body weights and body compositions were determined after 17 weeks on the high fat diet (58 kcal % fat). FATP5 KO mice were protected from adiposity, though no difference was observed in the lean mass between FATP5 KO animals and wild-type animals. See Table III.

TABLE III
Effect of FATP5 deletion on Body Composition after High Fat Diet

| | | Body | Lean | |
|---|---|---|---|---|
| Genotype | Diet | Weight (g) | Mass (g) | Fat Mass (g) |
| Wild-type | High Fat | 44.32 ± 1.62 | 26.26 ± 0.73 | 15.51 ± 1.18 |
| FATP5 -/- | High Fat | 39.24 ± 1.17* | 25.87 ± 0.61 | 11.34 ± 0.88** |
| Wild-type | Chow | 40.08 ± 0.93 | 28.15 ± 0.43 | 9.43 ± 0.81 |

*p < 0.05
**p < 0.01

### EXAMPLE 5

### Energy Expenditure in FATP5 Knock Out and Wild Time Mice Maintained on a High Fat Diet

Fourteen male FATP5 KO and wild type mice were maintained on a high fat diet as described in Example 3. Energy expenditure was determined using calorimetry analysis after 17 weeks on the high fat diet (58 kcal % fat). Results demonstrated FATP5 KO mice had significantly higher energy expenditure compared to wild-type mice. See Table IV.

TABLE IV
Mice without FATP5 showed higher oxygen consumption in fed state

| | | Oxygen consumption in | Oxygen consumption |
|---|---|---|---|
| Genotype | Diet | fasting state (ml/kg/h) | in fed state (ml/kg/h) |
| wild-type | High Fat | 813.30 ± 21.77 | 916.73 ± 11.53 |
| FATP5 -/- | High Fat | 844.44 ± 17.85 | 1006.61 ± 28.46* |

*p < 0.05

### EXAMPLE 6

### FATP5 Deletion Protects from Diet-Induced Insulin Resistance

High fat feeding induces insulin resistance in mice. In order to determine whether FATP5 protects against diet-induced insulin resistance a glucose tolerance test was preformed. Briefly, FATP5 KO and wild type mice were maintained on high fat diet (58 kcal % fat) or a standard law chow diet. Mice were fasted overnight and injected i.p. with 2mg/kg of glucose. Blood glucose levels were measuring at six intervals over a 180 minute period, using a glucometer (Bayer) according to the manufacturer's protocol. FATP5 KO animals maintained on a high fat diet had a glucose clearance profile similar to wild-type mice maintained on a chow diet, while the wild-type mice maintained on a high fat diet had a significantly impaired glucose clearance profile. See Table V.

TABLE V
FATP5 deletion protects from diet-induced insulin resistance

| Genotype | Diet | 0 min | 15 min | 30 min | 60 min | 120 min | 180 min | AUC |
|---|---|---|---|---|---|---|---|---|
| Wild type | High | 187.6 ± 10.1 | 407.5 ± 27.1 | 470.3 ± 25.2 | 411.7 ± 29.8 | 261.4 ± 33.2 | 169.1 ± 17.1 | 53,978 ± 2,630 |
| | Fat | | | | | | | |
| FATP5 -/- | High | 173.4 ± 10.9 | 381.3 ± 37.4 | 401 ± 37.9 | 312.4 ± 35.2* | 176.46 ± 17.3* | 136.6 + 11.8 | 36,230 ± 3,757*** |
| | Fat | | | | | | | |
| Wild type | Chow | 132.4 ± 5.3 | 380.4 ± 22.4 | 401.9 ± 22.6 | 369.6 ± 24.6 | 213.8 ± 19.8 | 147.4 ± 9.9 | 27,504 ± 5,260 |

*p < 0.05 compared to WT High Fat
***p < 0.001 compared to WT High Fat
All glucose values in mg/dl AUC (area under the curve) values in min * mg/dl.

### EXAMPLE 7

### FATP5 Deletion Animals Show a Beneficial Plasma Lipid Profile

FATP5 knock-out and wild type mice (n=14, 8 weeks of age) were fed a high-fat diet for 28 weeks. Age-matched wild type mice were maintained on a chow diet as a control. Serum was collected from the overnight fasted animals. Commercially available kits were used to measure triglycerides, free fatty acids, cholesterol, beta-hydroxybutyrate (all Sigma) as well as insulin and leptin (both CrystalChem). A trend towards decreased serum triglyceride levels was noted, and free fatty acid levels were significantly decreased in FATP5 knock-out animals. Examination of serum from overnight fasted animals showed few significant differences between KO and wild-type animals in other parameters, as serum cholesterol, ketone bodies (beta-hydroxy-butyrate) or lipoprotein profile (not shown) were unchanged in FATP5 knock-out animals. Thus, inhibition of FATP5 is likely to have no adverse effects, and may positively affect, the blood lipid profile. See Table V.

TABLE VI
FATP5 deletion demonstrate beneficial plasma lipid profile

| | | Glucose | Insulin | Free Fatty | Triglycerides |
|---|---|---|---|---|---|
| Genotype | Diet | (mg/dl) | (pg/ml) | Acids (mM) | (mg/dl) |
| wild-type | High Fat | 113.3 | 2057 ± 398 | 1.65 ± 0.1 | 201 ± 25 |
| FATP5 -/- | High Fat | 121.2 | 1189 ± 207 | 1.38 ± 0.08* | 159 ± 15.6 |
| wild-type | Chow | | 1504 ± 285 | 1.68 ± 0.1 | 180.4 ± 14.2 |

| | | | Ketone | | |
|---|---|---|---|---|---|
| | | Cholesterol | Bodies | Glycerol | Leptin |
| Genotype | Diet | (mg/dl) | (mg/dl) | (mg/dl) | (pg/ml) |
| wild-type | High Fat | 279.5 ± 22.9 | 9.39 ± 1.12 | 58.25 ± 4.81 | 51730 ± 2468 |
| FATP5 -/- | High Fat | 304.9 ± 30.9 | 6.86 ± 1.34 | 46.99 ± 6.91 | 28210 ± 1164** |
| wild-type | Chow | 200.5 ± 21.3 | 9.66 ± 1.2 | 51.24 ± 4.09 | 28767 ± 4070 |

*p < 0.05
**p < 0.01

### EXAMPLE 8

### FATP5 Knock Out Mice Display Lower Liver Lipid Levels

FATP5 knock-out and wild type mice were maintained on a high-fat diet for 28 weeks. Age-matched wild type mice were maintained on a chow diet as a control. Livers were removed after 28 weeks and liver lipids were measured as a % wet weight. FATP5 knock out animals had decreased liver lipid levels when compared to wild type control animals. This suggests protection from fatty liver disease. See Table VI.

TABLE VII
Effect of FATP5 deletion on Liver Lipid Contents

| | | liver lipids |
|---|---|---|
| Genotype | Diet | (% wet weight) |
| wild-type | High Fat | 16.55 ± 0.47 |
| FATP5 -/- | High Fat | 14.57 ± 0.47** |
| wild-type | Chow | 16.05 ± 0.37 |

**p < 0.01

### EXAMPLE 9

### FATP5 Deletion Results in a Small Increase in Fecal Fat, but no Significant Difference in Fecal Calories

FATP5 knock-out and wild type animals were maintained on a high fat diet as described above for 11 weeks. Feces was collected from the animals and fecal lipid levels were measured as a percent a dry weight. FATP5 knock out animals displayed a slight increase in fecal lipid levels. See Table VIII. We also exposed FATP5 knock-out animals to a high fat diet for 5 days, collected feces and measured total fecal calorie content by bomb calorimetry. Data indicate that there is no difference in total fecal calorie between wild type and FATP5 knock-out mice. See Table IX.

TABLE VIII
Effect of FATP5 deletion on Fat in the Feces

| Genotype | Fecal Lipid (% dry weight) |
|---|---|
| Wild-type | 5.86 ± 0.23 |
| FATP5 -/- | 6.68 ± 0.27 |

*p < 0.05

TABLE IX
Deletion of FATP5 did not change the total fecal calorie

| Genotype | Diet | Fecal calorie (cal/g) |
|---|---|---|
| wild-type | High Fat | 4039.38 ± 191.67 |
| FATP5 -/- | High Fat | 3902.66 ± 61.80 |
| wild-type | Chow | 3735.57 ± 63.94 |
| FATP5 -/- | Chow | 3755.57 ± 62.73 |

### EXAMPLE 10

### FATP5 Deletion Mice are Deficient in Conjugation of Recirculated Bile Acids and Show Increased De Novo Bile Acid Biosynthesis

Bile Acid Composition of FATP5 Deletion Mice

Bile was collected from fasted wild-type and FATP5 knock-out mice and bile acid composition was analyzed by LC-MS. Bile acid groups were identified using commercially available standards. Within each major bile acid group, the area under the curve (AUC) for individual bile acid peaks was determined, and identified as relative abundance of the major bile acid groups in wild-type and knock-out mice. While the total amount of bile acids was similar in both groups of mice, bile from FATP5 deletion mice contained primarily unconjugated bile acids, while most of the bile acids from wild-type mice were conjugated. In addition, FATP5 deletion mice had significantly fewer dihydroxylated bile acids, and a larger amount of tetrahydroxylated bile acids compared to wild-type mice. See Table X.

TABLE X
Bile Acid Composition of wild-type and FATP5 deletion mice

| | Relative | | | |
|---|---|---|---|---|
| | Abundance (AUC) | | % of Total | |
| Bile Acids | FATP5 -/- | wt | FATP5 -/- | wt |
| Unconjugated DiOH | 1,024,619 | 0 | 0.5 | 0.0 |
| Unconjugated TriOH | 135,778,358 | 8,111,524 | 65.9 | 5.2 |
| Unconjugated TetraOH | 37,553,543 | 0 | 18.2 | 0.0 |
| Taurine conjugated | 5,065,264 | 20,977,106 | 2.5 | 13.5 |
| DiOH | | | | |
| Taurine conjugated | 26,668,812 | 122,257,746 | 12.9 | 78.8 |
| TriOH | | | | |
| Taurine conjugated | 0 | 3,734,777 | 0.0 | 2.4 |
| TetraOH | | | | |
| Total | 206,090,596 | 155,081,153 | 100 | 100 |

Examination of individual taurine-conjugated dihydroxylated bile acids in wild-type and FATP5 knock-out mice demonstrated that this fraction in FATP5 deficient animals group consists entirely of taurochenodeoxycholatea, bile acid which can be produced by de novo synthesis. In contrast, two bile acid species which are generated during enterohepatic recirculation, but not through de novo synthesis, taurohyodeoxycholate and taurodeoxycholate, are detected in wild type animals, but are not detectable above background in the knock-out mice. See Table XI. Our data suggest that FATP5 is required for the conjugation of bile acids during enterohepatic recirculation, but not during de novo synthesis. Since de novo synthesis only accounts for a small percentage of the bile acids in bile, a defect in enterohepatic recirculation will result in a shift of the total bile acid pool towards unconjugated species.

TABLE XI
Identification of individual taurine conjugated dihydroxy bile acids

| | | Relative | |
|---|---|---|---|
| | | Abundance (AUC) | |
| Bile Acid | Source | FATP5 -/- | wt |
| Taurohyodeoxycholate | Recirculation | 0 | 7,277,127 |
| Taurochenodeoxycholate | De novo Synthesis | 5,065,264 | 5,275,366 |
| Taurodeoxycholate | Recirculation | 0 | 6,971,675 |

Gene Expression Analyses

To further examine the consequences of deletion of FATP5 we compared gene expression in the livers of wild-type and FATP5 deletion mice using a combination of transcriptional profiling and real-time RT-PCR analysis. Gene expression was measured by transcriptional profiling GeneChip analysis, performed according to manufacturer's directions (Affymetrix, Santa Clara, Calif.), or TaqMan quantitative PCR analysis, performed according to the manufacturer's directions (Perkin Elmer Applied Biosystems, Foster City, Calif.).

For transcriptional profiling experiments, total RNA was isolated from homogenized tissue by TriazolTM (Life Technologies, Inc.) following the manufacturer's recommendations. RNA was stored at 80° C. in diethyl pyrocarbonate-treated deionized water. Detailed methods for labeling the samples and subsequent hybridization to the arrays are available from Affymetrix (Santa Clara, Calif.). Briefly, 5.0 μg of total RNA was converted to double-stranded cDNA (Superscript; Life Technologies, Inc.) priming the first-strand synthesis with a T7-(dT)24 primer containing a T7 polymerase promoter (Affymetrix Inc.). All of the double-stranded cDNA was subsequently used as a template to generate biotinylated cRNA using the incorporated T7 promoter sequence in an in vitro transcription system (Megascript kit; Ambion and Bio-11-CTP and Bio-16-UTP; Enzo). Control oligonucleotides and spikes were added to 10 μg of cRNA, which was then hybridized to Mu U74 oligonucleotide arrays for 16 h at 45° C. with constant rotation. The arrays were then washed and stained on an Affymetrix fluidics station using the EUKGE-WS1 protocol and scanned on an Affymetrix GeneArray scanner. Data analysis was performed using MAS 5.0 software.

For real time PCR experiments, total RNA was also prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control 18S gene, confirming efficient removal of genomic DNA contamination. PCR probes were designed by PrimerExpress software (Perkin Elmer Applied Biosystems) based on the relevant gene sequence. To standardize the results between the different tissues, two probes, distinguished by different fluorescent labels, were added to each sample. The differential labeling of the probe for the gene and the probe for 18S RNA (as an internal control) thus enabled their simultaneous measurement in the same well. Forward and reverse primers and the probes for both 18S RNA and the gene of interest were added to the TaqMan Universal PCR Master Mix (PE Applied Biosystems). Although the final concentration of primer and probe could vary, each was internally consistent within a given experiment. A typical experiment contained 200 nM each of the forward and reverse primers and 100 nM of the probe for the 18S RNA, as well as 600 nM of each of the forward and reverse primers and 200 nM of the probe for the gene of interest. TaqMan matrix experiments were carried out using an ABI PRISM 770 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds, followed by 60° C. for 1 minute.

The following method was used to quantitatively calculate gene expression in the tissue samples, relative to the 18S RNA expression in the same tissue. The threshold values at which the PCR amplification started were determined using the manufacturer's software. PCR cycle number at threshold value was designated as CT. Relative expression was calculated as 2^{-((CTtest-CT18S) tissue of interest-(CTtest-CT18S) lowest expressing tissue in panel)}. Samples were run in duplicate and the averages of 2 relative expression levels that were linear to the amount of template cDNA with a slope similar to the slope for the internal control 18S were used. Fold increase in expression was determined as relative expression in FATP5 deletion animals as compared to relative expression levels in wild-type control animals.

Upregulation of both the cholesterol and bile acid biosynthetic pathway related genes was found. Several target genes of FXR, a nuclear hormone receptor which is known to be activated by bile acids (Parks et al, Science 284:1365-8, 1999), are regulated in a manner suggesting decreased activity of FXR. Since FXR is activated strongly by dihydroxy bile acids, but is almost insensitive to trihydroxy bile acids (Parks et al, Science 284:1365-8, 1999), decreased FXR activity is consistent with decreased levels of dihydroxybile acids found in FATP5 knock-out animals. See Table XII.

Upregulation of both cholesterol biosynthesis as well as LDL-R, a gene involved in cholesterol uptake could be an adaptive response which allows intracellular cholesterol levels to remain normal in the presence of increased bile acid synthesis. We see little regulation of fatty acid oxidation genes in the knock-out animals, consistent with the finding that the respiratory exchange ration is unchanged in FATP5 deletion animals. In addition, we see strong downregulation of key genes involved in gluconeogenesis, one explanation for improved insulin sensitivity of FATP5 deletion animals. See Table XII.

TABLE XII
Gene expression in livers of FATP5 deletion mice

Fold increase in gene expression
in FATP5 -/- animals

Cholesterol Biosynthesis

| | |
|---|---|
| HMG-CoA-Synthase 1 | 12.5 |
| HMG-CoA-Reductase | 5.9 |
| di-phosphomevalonate | 4.0 |
| decarboxylase | |
| IPP isomeraes | 14.3 |
| Farnesyl-pyrophosphate synthase | 7.1 |
| Squalene synthetase | 4.5 |
| Squalene monooxygenase | 8.3 |
| Lathosterol oxidase | 3.1 |
| 7-Dehydro-cholesterol reductase | 3.3 |
| Lanosterol-14-alpha-demethylase | 6.7 |

Bile Acid Biosynthesis

| | |
|---|---|
| C7alpha hydroxylase | 7.2 |
| alpha methylacyl-CoA racemase | 5.9 |

Cholesterol uptake

| | |
|---|---|
| LDL-R | 1.6 |

FXR Target Genes

| | |
|---|---|
| SHP | 0.6 |
| PLTP | 4.0 |
| C7alpha hydroxylase | 7.2 |

Faox

| | |
|---|---|
| CPT1 | 1.1 |
| Acyl-CoA-oxidase (peroxisomal) | 0.8 |
| SCAD | 0.8 |
| MCAD | 0.7 |
| LCAD | 0.8 |
| PPARalpha | 0.7 |

Insulin regulated pathway/Gluconeogenesis

| | |
|---|---|
| PEPCK | 0.8 |
| G6Pase | 0.3 |
| IGFBP1 | 0.3 |
| SREBP1 | 0.4 |

Lipogenesis

| | |
|---|---|
| FAS | 0.9 |
| SCD1 | 0.9 |
| Transketolase | 1.0 |

### EXAMPLE 11

### Acyl-CoA Ligase Activity by FATP5

Expression and Purification of hsFATP5:

The human FATP5 coding sequence containing a C-terminal 6x-HIS tag was inserted into the pFastBAC vector and Baculovirus expressing hsFATP5-His was constructed using established methods. Sf9 insect cells were infected with hsFATP5-His containing baculovirus, harvested 48 hours later and frozen in liquid nitrogen. Cells were thawed on ice in lysis buffer (50 mM Tris, pH 8.0, 20 mM imidazole, 1 M NaCl, 0.5% n-dodecyl-maltoside, 10% glycerol, 1 mM 2-Mercapto-ethanol containing a standard cocktail of protease inhibitors) and lysed by sonication. The lysate was spun at 17,700 g for 30 minutes at 4° C. and the supernatant applied to a Ni-NTA superflow resin (Qiagen) equilibrated in lysis buffer, 500 μl resin was used per 0.5 1 of cells. The resin was incubated at 4° C. for 1 hour with gentle shaking, centrifuged at 400 g for 15 minutes at 4° C., and the resin transferred into a 10 ml Biorad disposable column. The column was washed with 40 column volumes of equilibration buffer, and bound material was eluted by adding 2 column volumes of elution buffer-(50 mM Tris, pH 8.0, 250 mM imidazole, 1 M NaCl, 0.5% n-dodecyl-maltoside, 10% glycerol, 1 mM 2-Mercapto-ethanol containing a standard cocktail of protease inhibitors). hsFATP5 was the major band in each elution fraction and was approximately 70% pure as judged by Code Blue (Pierce Chemicals) staining. The identity of the major band as hsFATP5 was confirmed by MALDI-TOF analysis of tryptic digests and N-terminal microsequencing. The eluted material was stored at −20° C. Western blotting showed that, while FATP5 can be purified using this method, significant amounts of hsFATP5 are also present in the flow-through fraction.

The human FATP5 coding sequence containing a N-terminal GST tag was inserted into the pFastBAC vector and Baculovirus expressing GST-hsFATP5 was constructed using established methods. Sf9 insect cells were infected with hsGST-FATP5 containing baculovirus, harvested 48 hours later and frozen in liquid nitrogen. Cells were thawed and protein purified as described above for hsFATP5-His, with the exception that a glutathione sepharose 4B resin was employed for purification and protein was eluted with glutathione elution buffer (50 mM Tris, pH 8.0, 50 mM glutathione, 1 M NaCl, 0.5% n-dodecyl-maltoside, 10% glycerol, 1 mM 2-Mercapto-ethanol containing a standard cocktail of protease inhibitors).

FATP5 Acyl-CoA ligase activity can be measured by any known method in the art. Included here are exemplification of enzyme activity determinations using a crude assay, a malachite green assay, or a mass spectrometry analysis.

Crude Acyl-CoA Ligase Activity Assay

Acyl-CoA ligase activity assays were performed using the modified method of Kelley and Vessey ((1994) Biochem J. 304:945-949). Briefly, 50 μg of crude supernatant, or 2 μg of column-purified material was incubated at 30° C. with 100 mM Tris, pH 7.4, 10 μM CoA, 10 μM Cholate, 15 mM MnCl₂, 1 mM ATP in a volume of 0.5 mL. The reaction was terminated by the addition of 0.05 mL of 0.1 M EDTA, pH 7.5, and 0.2 m]L of 60 mM succinic acid. Unreacted cholate was extracted with Dole's reagent (isopropyl alcohol, heptane, 1 M H₂SO₄; 40:10:1 by volume). After vigorous mixing, the protein was removed by centrifugation, and 650 μl of the supernatant was extracted with 350 μl of heptane plus 190 μl of a solution containing 400 nM Mops-NaOH (pH 6.5). The lower (aqueous) layer was washed three times with 400 μl of heptane, and the radioactivity in that fraction was measured by scintillation counter. Activity was expressed as pmol/min/mg protein.

Malachite Green Acyl-CoA Ligase Activity Assay

FATP5 activity can also be measured using malachite green as a detection method. The reaction mix contains the following components: 50 mM Tris, pH 7.5, 60 μM bile of fatty acid, 50 μM coenzyme A, 0.625 U/ml inorganic pyrophosphatase, 1 mM MgCl₂, 120 uM ATP, and 2 μg of total protein from a FATP5 crude membrane preparation or column purification. Reactions are quenched with EDTA (0.1 M final concentration) and 50 μL of malachite green solution (0.45 grams malachite green, 7 grams ammonium molybdate, 1.33 N HCl, 1 L final volume) is added for phosphate detection. Absorbance at 610 nm is measured on a Spectramax 384 plate reader 30 minutes after malachite green addition.

Acyl-CoA Ligase Activity Assay Using LC/MS

Still further, FATP5 enzymatic activity can be measured by direct detection of the product, acyl-CoA using a modified version of the LC/MS assay developed by Ikegawa et al. ((1999) Anal. Biochem. 266; 125-132) The following is a description of a FATP5 LC/MS assay using cholate and chenodeoxycholate as bile acid substrates. Fatty acids were also used as substrates.

The reaction mix contains the following components: 50 mM Tris, pH 7.5, 60 uM bile acid (or fatty acid), 50 uM coenzyme A, 1 mM DTT, 0.42 U/ml inorganic pyrophosphatase, 1 mM MgCl₂, 120 uM ATP, and 2 μg of total protein from a FATP5 purification (described above). The reaction is initiated by the addition of substrates and is quenched with acetic acid (final concentration=0.63%). The quenched reaction (50 μL) is injected on an Agilent 1100 HPLC system fitted with a single quadrapole mass spectrometer (G1946D SL model). A gradient from 5-95% Acetonitrile (organic or B phase) is run at a flow rate of 0.5 mL/min over 18 minutes (the aqueous or A phase is 10 mM Ammonium acetate). The total run time is 20 minutes consisting of a 1 min equilibration initially and a 1 min equilibration after the gradient is complete. The following mass-to-charge ratios are monitored in SIM (single ion monitoring) mode: cholate (407.6) cholyl-CoA (1156.5, 577.5), chenodeoxycholate (1141, 569) and CoA (766).

Material from FATP5 crude membrane preparations demonstrated 0.06 uM/min acyl-CoA ligase activity, while purified protein fractions demonstrated an acyl-CoA ligase activity of 0.09 uM/min. In contrast, material from insect cells expressing an unrelated protein did not show any activity in the purified fractions. These data provide evidence that CoA-ligase activity is associated with FATP5 protein and may be used to assay FATP5 protein function.

The assays described herein, or other related assays known in the art for assessment of acyl-CoA ligase activity can be modified to accommodate high throughput screening of candidate FATP5 inhibitor compounds.

### EXAMPLE 12

### Screening for an Agent that Modulates Bile or Fatty Acid Uptake

Human embryonic kidney 293 cells stably transfected with pIRES-mFATP5 vector were cultured and passed in DMEM base medium. 8×10⁴ cells per well were plated in a 96-well microtiter plate, and washed with washing buffer (20 mM HEPES, 4.2 mM NaHCO3 in IX Hanks basic salt solution). Inhibitor in 1 mM taurocholate in washing buffer is then added to the cells at 37° C. The uptake assay is initiated by addition of 5 mM final BODIPY-fatty acid (4,4-difluoro-5-methyl-4-bora-3a,4a-diaza-s-indacene-dodecanoic acid). The uptake assay is run for 15-60 minutes, and stopped by washing with 0.1% bovine serum albumin in washing buffer. The amount of BODIPY-fatty acid taken up by the cells is then determined by measuring the fluorescence of the cells after washing (excitation=450 nm, emission=530 nm). Inhibition of uptake as a function of concentration of inhibitor is graphed and fit to the equation (Y=max+(max−min)/(1+10\hat{ }((log IC50-X)*HillSlope)) to calculate IC50 value for the inhibitor.

While this invention has been particularly shown and described with references to preferred embodiments thereof, it will be understood by those skilled in the art that various changes in form and details may be made therein without departing from the scope of the invention encompassed by the following claims.

1. A method of identifying whether a compound is a candidate compound capable of modulating an FATP5-mediated metabolic disorder, the method comprising:
a. combining a test compound with a composition comprising a FATP5 polypeptide;
b. measuring an acyl-CoA ligase activity of the FATP5 polypeptide in the composition in the presence and absence of the test compound
c. identifying the test compound as the candidate compound for use in modulating the FATP5-mediated metabolic disorder when the FATP5 polypeptide acyl-CoA ligase activity in the presence of the compound differs from the FATP5 polypeptide Acyl-CoA ligase activity in the absence of the compound;
wherein the FATP5 mediated metabolic disorder is selected from the group consisting of body weight, insulin resistance, dyslipidemia, fatty liver disease and cardiovascular disease.

2. The method of claim 1, wherein the composition comprising the FATP5 polypeptide is selected from the group consisting of a cell expressing the FATP5 polypeptide, a membrane preparation comprising the FATP5 polypeptide, and an isolated FATP5 polypeptide.

3. The method of claim 1 wherein the acyl-CoA ligase activity of the FATP5 polypeptide is determined by measuring an activity selected from the group consisting of:
a. production of phosphate, acyl-CoA, or AMP;
b. consumption of coenzyme A, ATP, or fatty acid or bile acid; and
c. fatty acid or bile acid uptake.

4. The method of claim 1 wherein the acyl-CoA ligase activity is a bile acid CoA ligase activity.

5. A method of identifying whether a compound is a candidate compound capable of modulating an FATP5-mediated metabolic disorder, the method comprising:
a. combining a test compound with a composition comprising a FATP5 polypeptide;
b. determining whether the test compound binds to the FATP5 polypeptide;
c. administering a compound identified as binding to the FATP5 polypeptide in step (b) to a mammal;
d. determining whether the test compound modulates an FATP5 mediated process;
e. identifying the test compound that modulates an FATP5 mediated process in step (d) as a candidate compound useful for modulating an FATP5 mediated disorder;
wherein the FATP5 mediated process is selected from the group consisting of:
body weight, body fat composition, feeding behavior, insulin resistance, glucose uptake, hepatic glucose output, fatty acid uptake, bile acid uptake, hepatic lipid content, bile acid composition in plasma, liver, bile or feces, dyslipidemia, and plasma lipid composition;
and wherein the FATP5 mediated metabolic disorder is selected from the group consisting of body weight, insulin resistance, dyslipidemia, fatty liver disease and cardiovascular disease.

6. The method of claim 5, wherein the composition comprising the FATP5 polypeptide is selected from the group consisting of a cell expressing the FATP5 polypeptide, a membrane preparation comprising the FATP5 polypeptide, and an isolated FATP5 polypeptide.

7. The method of claim 1 further comprising:
c. administering the test compound identified as modulating FATP5 Acyl-CoA ligase activity to a mammal;
d. determining whether the test modulates an FATP5 mediated process;
e. identifying the test compound that modulates an FATP5 mediated process in step (d) as a candidate compound useful for modulating an FATP5 mediated disorder;
wherein the FATP5 mediated process is selected from the group consisting of:
body weight, body fat composition, feeding behavior, insulin resistance, glucose uptake, hepatic glucose output, fatty acid uptake, bile acid uptake, hepatic lipid content, bile acid composition in plasma, liver, bile or feces, dyslipidemia, and plasma lipid composition.

8. A method of identifying whether a compound is a candidate compound capable of modulating an FATP5-mediated metabolic disorder, the method comprising:
a. combining a test compound with a composition comprising a cell capable of expressing an FATP5 polypeptide;
b. measuring expression of the FATP5 polypeptide in the composition in the presence and absence of the test compound
c. identifying the test compound as the candidate compound for use in modulating the FATP5-mediated metabolic disorder when the FATP5 polypeptide expression in the presence of the compound differs from the FATP5 polypeptide expression in the absence of the compound;
wherein the FATP5 mediated metabolic disorder is selected from the group consisting of body weight, insulin resistance, dyslipidemia, fatty liver disease and cardiovascular disease.

9. The method of claim 8 wherein the FATP5 expression is determined by measuring any one of transcript level, protein level, fatty acid or bile acid uptake activity, or acyl CoA ligase activity.

10. A method of treating a FATP5 mediated metabolic disorder in an individual comprising administering to the individual an effective amount of an agent that inhibits FATP5 activity.

11. The method of claim 10 wherein the FATP5-mediated metabolic disorder is selected from the group of obesity, insulin resistance, type 2 diabetes, dyslipidemia, fatty liver disease, and cardiovascular disease.

12. The method of claim 11 wherein dyslipidemia is selected from the group consisting of elevated serum triglyceride levels, elevated bile acid levels, and elevated free fatty acid levels.

13. The method of claim 11 wherein cardiovascular disease is selected from the group consisting of coronary heart disease, atherosclerosis, hypertension, and ischemic heart disease.

14. A method of identifying whether a compound is a candidate compound capable of modulating an FATP5-mediated metabolic disorder, comprising:
a. combining a test compound and a fatty acid or bile acid with a test cell expressing a FATP5 polypeptide;
b. measuring uptake of the fatty acid or bile acid in the test cell; and
c. identifying the test compound as a candidate compound for use in modulating the FATP5 mediated metabolic disorder when uptake of the fatty acid or bile acid into the test cell in the presence of the compound differs from the uptake of the fatty acid or bile acid into the test cell in the absence of the compound;
wherein the FATP5-mediated metabolic disorder is selected from the group of obesity, insulin resistance, type 2 diabetes, dyslipidemia, fatty liver disease and cardiovascular disease.

15. The method of claim 14 wherein dyslipidemia is selected from the group consisting of elevated serum triglyceride levels, elevated bile acid levels, and elevated free fatty acid levels.

16. The method of claim 14 wherein cardiovascular disease is selected from the group consisting of coronary heart disease, atherosclerosis, hypertension, and ischemic heart disease.


SEQUENCE LISTING (4 sequences)

SEQ ID NO: 1 - DNA, 1989 bp, mouse
  CDS (1)...(1989)
atggctctggccctgcgttggttcctgggagaccccacatgccttgtgctgcttggcttggcattgctgggcagaccctggatcagctcctggatgccccactggctgagcctggtaggagcagctcttaccttattcctattgcctctacagccacccccagggctacgctggctgcataaagatgtggctttcaccttcaagatgcttttctatggcctaaagttcaggcgacgccttaacaaacatcctccagagacctttgtggatgctttagagcggcaagcactggcatggcctgaccgggtggccttggtgtgtactgggtctgagggctcctcaatcacaaatagccagctggatgccaggtcctgtcaggcagcatgggtcctgaaagcaaagctgaaggatgccgtaatccagaacacaagagatgctgctgctatcttagttctcccgtccaagaccatttctgctttgagtgtgtttctggggttggccaagttgggctgccctgtggcctggatcaatccacacagccgagggatgcccttgctacactctgtacggagctctggggccagtgtgctgattgtggatccagacctccaggagaacctggaagaagtccttcccaagctgctagctgagaacattcactgcttctaccttggccacagctcacccaccccgggagtagaggctctgggagcttccctggatgctgcaccttctgacccagtacctgccagccttcgagctacgattaagtggaaatctcctgccatattcatctttacttcagggaccactggactcccaaagccagccatcttatcacatgagcgggtcatacaagtgagcaacgtgctgtccttctgtggatgcagagctgatgatgtggtctatgacgtcctacctctgtaccatacgatagggcttgtccttggattccttggctgcttacaagttggagccacctgtgtcctggcccccaagttctctgcctcccgattctgggctgagtgccggcagcatggcgtaacagtgatcttgtatgtgggtgaaatcctgcggtacttgtgtaacgtccctgagcaaccagaagacaagatacatacagtgcgcttggccatgggaactggacttcgggcaaatgtgtggaaaaacttccagcaacgctttggtcccattcggatctgggaattctacggatccacagagggcaatgtgggcttaatgaactatgtgggccactgcggggctgtgggaaggaccagctgcatccttcgaatgctgactccctttgagcttgtacagttcgacatagagacagcagagcctctgagggacaaacagggtttttgcattcctgtggagccaggaaagccaggacttcttttgaccaaggttcgaaagaaccaacccttcctgggctaccgtggttcccaggccgagtccaatcggaaacttgttgcgaatgtacgacgcgtaggagacctgtacttcaacactggggacgtgctgaccttggaccaggaaggcttcttctactttcaagaccgccttggtgacaccttccggtggaagggcgaaaacgtatctactggagaggtggagtgtgttttgtctagcctagacttcctagaggaagtcaatgtctatggtgtgcctgtgccagggtgtgagggtaaggttggcatggctgctgtgaaactggctcctgggaagacttttgatgggcagaagctataccagcatgtccgctcctggctccctgcctatgccacacctcatttcatccgtatccaggattccctggagatcacaaacacctacaagctggtaaagtcacggctggtgcgtgagggttttgatgtggggatcattgctgaccccctctacatactggacaacaaggcccagaccttccggagtctgatgccagatgtgtaccaggctgtgtgtgaaggaacctggaatctctga

SEQ ID NO: 2 - protein, 662 aa, mouse
MALALRWFLGDPTCLVLLGLALLGRPWISSWMPHWLSLVGAALTLFLLPLQPPPGLRWLHKDVAFTFKMLFYGLKFRRRLNKHPPETFVDALERQALAWPDRVALVCTGSEGSSITNSQLDARSCQAAWVLKAKLKDAVIQNTRDAAAILVLPSKTISALSVFLGLAKLGCPVAWINPHSRGMPLLHSVRSSGASVLIVDPDLQENLEEVLPKLLAENIHCFYLGHSSPTPGVEALGASLDAAPSDPVPASLRATIKWKSPAIFIFTSGTTGLPKPAILSHERVIQVSNVLSFCGCRADDVVYDVLPLYHTIGLVLGFLGCLQVGATCVLAPKFSASRFWAECRQHGVTVILYVGEILRYLCNVPEQPEDKIHTVRLAMGTGLRANVWKNFQQRFGPIRIWEFYGSTEGNVGLMNYVGHCGAVGRTSCILRMLTPFELVQFDIETAEPLRDKQGFCIPVEPGKPGLLLTKVRKNQPFLGYRGSQAESNRKLVANVRRVGDLYFNTGDVLTLDQEGFFYFQDRLGDTFRWKGENVSTGEVECVLSSLDFLEEVNVYGVPVPGCEGKVGMAAVKLAPGKTFDGQKLYQHVRSWLPAYATPHFIRIQDSLEITNTYKLVKSRLVREGFDVGIIADPLYILDNKAQTFRSLMPDVYQAVCEGTWNL

SEQ ID NO: 3 - DNA, 2310 bp, human
  CDS (89)...(2158)
cttaaaggcaggtagttagggctttggagcactcatcagagctaagagagattacacgctctcatctacttcagaaagagccaatgccatgggtatttggaagaaactaaccttactgctgttgctgcttctgctggttggcctggggcagcccccatggccagcagctatggctctggccctgcgttggttcctgggagaccccacatgccttgtgctgcttggcttggcattgctgggcagaccctggatcagctcctggatgccccactggctgagcctggtaggagcagctcttaccttattcctattgcctctacagccacccccagggctacgctggctgcatatagatgtggctttcaccttcaagatgcttttctatggcctaaagttcaggcgacgccttaacaaacatcctccagagacctttgtggatgctttagagcggcaagcactggcatggcctgaccgggtggccttggtgtgtactgggtctgagggctcctcaatcacaaatagccagctggatgccaggtcctgtcaggcagcatgggtcctgaaagcaaagctgaaggatgccgtaatccagaacacaagagatgctgctgctatcttagttctcccgtccaagaccatttctgctttgagtgtgtttctggggttggccaagttgggctgccctgtggcctggatcaatccacacagccgagggatgcccttgctacactctgtacggagctctggggccagtgtgctgattgtggatccagacctccaggagaacctggaagaagtccttcccaagctgctagctgagaacattcactgcttctaccttggccacagctcacccaccccgggagtagaggctctgggagcttccctggatgctgcaccttctgacccagtacctgccagccttcgagctacgattaagtggaaatctcctgccatattcatctttacttcagggaccactggactcccaaagccagccatcttatcacatgagcgggtcatacaagtgagcaacgtgctgtccttctgtggatgcagagctgatgatgtggtctatgacgtcctacctctgtaccatacgatagggcttgtccttggattccttggctgcttacaagttggagccacctgtgtcctggcccccaagttctctgcctcccgattctgggctgagtgccggcagcatggcgtaacagtgatcttgtatgtgggtgaaatcctgcggtacttgtgtaacgtccctgagcaaccagaagacaagatacatacagtgcgcttggccatgggcaatggacttcgggcaaatgtgtggaaaaacttccagcaacgctttggtcccattcggatctgggaattctacggatccacagagggcaatgtgggcttaatgaactatgtgggccactgcggggctgtgggaaggaccagctgcatccttcgaatgctgactccctttgagcttgtacagttcgacatagagacagcagagcctctgagggacaaacagggtttttgcattcctgtggagccaggaaagccaggacttcttttgaccaaggttcgaaagaaccaacccttcctgggctaccgtggttcccaggccgagtccaatcggaaacttgttgcgaatgtacgacgcgtaggagacctgtacttcaacactggggacgtgctgaccttggaccaggaaggcttcttctactttcaagaccgccttggtgacaccttccggtggaagggcgaaaacgtatctactggagaggtggagagtgttttgtctagcctagacttcctagaggaagtcaatgtctatggtgtgcctgtgccagggtgtgagggtaaggttggcatggctgctgtgaaactggctcctgggaagacttttgatgggcagaagctataccagcatgtccgctcctggctccctgcctatgccacacctcatttcatccgtatccaggattccctggagatcacaaacacctacaagctggtaaagtcacgactggtgcgtgagggttttgatgtggggatcattgctgaccccctctacatactggacaacaaggcccagaccttccggagtctgatgccagatgtgtaccaggctgtgtgtgaaggaacctggaagctctgaccacctagccaactggaaggcaatccaaaagtgtagagattgacactagtcagcttcacaaagttgtccgggttccagatgcccatggcccagtagtacttagagaataaacttgaatgtgtaaaaaaaaaaaaaaaaaaaaaaaaaaaaaa

SEQ ID NO: 4 - protein, 689 aa, human
MGIWKKLTLLLLLLLLVGLGQPPWPAAMALALRWFLGDPTCLVLLGLALLGRPWISSWMPHWLSLVGAALTLFLLPLQPPPGLRWLHIDVAFTFKMLFYGLKFRRRLNKHPPETFVDALERQALAWPDRVALVCTGSEGSSITNSQLDARSCQAAWVLKAKLKDAVIQNTRDAAAILVLPSKTISALSVFLGLAKLGCPVAWINPHSRGMPLLHSVRSSGASVLIVDPDLQENLEEVLPKLLAENIHCFYLGHSSPTPGVEALGASLDAAPSDPVPASLRATIKWKSPAIFIFTSGTTGLPKPAILSHERVIQVSNVLSFCGCRADDVVYDVLPLYHTIGLVLGFLGCLQVGATCVLAPKFSASRFWAECRQHGVTVILYVGEILRYLCNVPEQPEDKIHTVRLAMGNGLRANVWKNFQQRFGPIRIWEFYGSTEGNVGLMNYVGHCGAVGRTSCILRMLTPFELVQFDIETAEPLRDKQGFCIPVEPGKPGLLLTKVRKNQPFLGYRGSQAESNRKLVANVRRVGDLYFNTGDVLTLDQEGFFYFQDRLGDTFRWKGENVSTGEVESVLSSLDFLEEVNVYGVPVPGCEGKVGMAAVKLAPGKTFDGQKLYQHVRSWLPAYATPHFIRIQDSLEITNTYKLVKSRLVREGFDVGIIADPLYILDNKAQTFRSLMPDVYQAVCEGTWKL